(19) United States
(12) Patent Application Publication
(10) Pub. No.: US 2025/0325599 A1
(43) Pub. Date: Oct. 23, 2025

(54) A CLINICAL ISOLATED AVIRULENT STRAIN PROTECTS AGAINST WILDTYPE VIRULENT CLOSTRIDIOIDES DIFFICILE INFECTION

(71) Applicant: THE UNIVERSITY OF CHICAGO, Chicago, IL (US)

(72) Inventors: Eric PAMER, Chicago, IL (US); Qiwen DONG, Chicago, IL (US)

(73) Assignee: THE UNIVERSITY OF CHICAGO, Chicago, IL (US)

(21) Appl. No.: 18/870,325

(22) PCT Filed: May 31, 2023
(86) PCT No.: PCT/US2023/067674

§ 371 Date: Nov. 27, 2024

Related U.S. Application Data
(60) Provisional application No. 63/347,268, filed on May 31, 2022.
(60) Provisional application No. 63/359,594, filed on Jul. 8, 2022.
(60) Provisional application No. 63/359,410, filed on Jul. 8, 2022.

(51) Int. Cl. A61K 35/74; A61K 39/00; A61K 39/02; A61K 45/06; A61P 31/04; C12N 1/20; C12N 7/00; C12N 9/22; C12N 15/11; C12Q 1/6809; C12Q 1/686; C12Q 1/689; C12R 1/01

(52) U.S. Cl. CPC A61K 35/74; A61K 39/0208; A61K 45/06; A61P 31/04; C12N 1/20; C12N 7/00; C12N 9/222; C12N 15/111; C12Q 1/6809; C12Q 1/686; C12Q 1/689; A61K 2039/522; C12N 2310/20; C12N 2795/14021; C12N 2795/14041; C12R 2001/01

(57) ABSTRACT

Aspects herein concern cdtR mutations that can lead to reduced or absent virulence in C. difficile bacteria. Disclosed are compositions and methods using the cdtR mutants to treat or prevent C. difficile infections or diseases caused by C. difficile. Also disclosed are methods for detecting or determining C. difficile virulence based on mutation status of the cdtR gene in C. difficile.

### CROSS-REFERENCE TO RELATED APPLICATIONS

This application claims the benefit of priority to U.S. Provisional Patent Application Ser. No. 63/347,268, filed May 31, 2022, U.S. Provisional Patent Application Ser. No. 63/359,410, filed Jul. 8, 2022, and U.S. Provisional Patent Application Ser. No. 63/359,594, filed on Jul. 8, 2022, each of which are hereby incorporated by reference in their entirety.

This invention was made with government support under grant number AI095706 awarded by the National Institutes of Health. The government has certain rights in the invention.

### BACKGROUND OF THE INVENTION

### I. Sequence Listing

The instant application contains a Sequence Listing which has been submitted in XML format and is hereby incorporated by reference in its entirety. Said XML copy, created on May 30, 2023, is named “ARCD.P0771WO—Sequence Listing” and is 16.524 kilobytes in size.

### II. Field of the Invention

This invention relates to the field of microbiology, medicine, and pathology.

### III. Background

Clostridioides difficile is a Gram-positive, spore-forming anaerobic bacterium, and the leading cause of nosocomial infections in the United States.¹⁻³ Infections are acquired by oral ingestion of C. difficile spores, which are prevalent in the environment and can survive for extended periods of time on contaminated surfaces. Upon ingestion, C. difficile spores germinate, produce toxins and cause colitis and, in severe cases, can result in mortality. The major virulence factors of C. difficile are toxins A (tcdA) and B (tcdB), which are encoded in the Pathogenicity Locus (PaLoc).^{4,5} These toxins glycosylate and thereby inactivate host GTPases, triggering the death of intestinal epithelial cells and lead to gut inflammation.⁶

C. difficile species is comprised of hundreds of strain types across more than 6 phylogenetic clades. PCR- and sequencing-based approaches, including PCR-ribotyping (RT) and multilocus sequencing typing (MLST/ST), have been used to characterize C. difficile strain types. More recently, whole-genome sequencing (WGS) has greatly contributed to the understanding of C. difficile diversity, evolution, and epidemiology.⁷ Almost two decades ago, the BI/NAP1/027 strain, characterized as ST1 by MLST, emerged as a cause of severe nosocomial outbreaks and increased C. difficile infection (CDI) incidence in North America and Europe. Since then, the prevalence of ST1 has declined but it remains among the most frequently isolated strains in hospital and community-acquired CDI cases in the US.^{2,8-11} The ST1 C. difficile strain encodes the additional CDT toxin (encoded by cdtA and cdtB and also referred to as binary toxin), which is an ADP-ribosyltransferase that modifies actin and disrupts cellular cytoskeleton organization.¹² The ST1 strains have higher MICs to several antibiotics, most notably fluroquinolones, and produce higher amounts of toxins A and B compared to non-ST1 strains.^{13,14} The relative virulence of the ST1 strain is controversial, however, with some studies demonstrating clinical disease severities similar to other strains.¹⁵⁻¹⁷ Host factors can impact the severity of CDI, including underlying diseases, immune competence and microbiome composition.^{18,19} Whether genetic variants of ST1 explain diverse disease manifestations is unknown.

### SUMMARY OF THE INVENTION

Aspects of the disclosure relate to the discovery that mutations in the cdtR gene, including a 69-base pair, in-frame deletion (such as SEQ ID NO:9), can reduce or remove virulence of bacteria, including C. difficile. The discovery can be used to modify virulent strains of bacteria to reduce their capability to infect a patient and/or reduce the bacteria capability to cause intestinal disorders such as colitis. The discovery also allows for the administration of bacteria having a cdtR mutant, including SEQ ID NO:8, or a bacteriophage encoding the cdtR mutant, to prevent or treat a C. difficile pathology. The discovery also allows for the determining the virulence of C. difficile present in a patient, and for determining a therapeutic regime for treating the patient based on the virulence of the C. difficile present in the patient.

Aspects of the disclosure relate to a method for treating and/or preventing bacterial pathogenesis, such as C. difficile pathogenesis. Further aspects of the disclosure relate to a method of analyzing virulence of a bacteria strain present in an infection, including an infection in a patient. Yet further aspects relate to treating a bacterial infection in a patient where a cdtR gene, including a wild-type and/or mutant cdtR gene, have been identified in the bacterial infection. Yet further aspects relate to C. difficile cells, including engineered C. difficile cells, that comprise a mutant cdtR gene. Yet further aspects relate to C. difficile cells, including engineered C. difficile cells, that express a mutant cdtR protein. Yet further aspects relate to C. difficile cells, including engineered C. difficile cells, that do not express a functional cdtR protein. Yet further aspects relate to C. difficile cells, including engineered C. difficile cells, that express reduced levels of cdtR protein as compared to wild type C. difficile. Other aspects include compositions comprising at least one of the C. difficile cells disclosed herein. Other aspects include compositions comprising at least two of the C. difficile cells disclosed herein. Yet further aspects relate to bacteriophage, which may be engineered bacteriophage, that encode a mutant cdtR gene. The bacteriophage may infect a bacterial cell, including a C. difficile bacterial cell, and cause reduced levels of functional cdtR protein and/or reduce the downstream effects of the cdtR protein.

Aspects of the disclosure include various methods directed to a patient including methods for treating and/or preventing C. difficile pathogenesis, treating and/or preventing a C. difficile infection, treating and/or preventing colitis, treating and/or preventing intestinal damage, reducing the severity of a bacterial infection, reducing the virulence of a bacterial infection, reducing the virulence of a bacterium any of which may include 1, 2, 3, 4, 5, 6, or more steps including any of the following: administering to a patient a C. difficile cell having a reduced cdtR protein expression relative to a wild type C. difficile, administering to a patient a C. difficile cell that expresses a mutant cdtR protein, administering to a patient a C. difficile cell that does not express a functional cdtR protein, administering to the patient one or more antibiotics, including after administering the C. difficile cell, administering a therapeutic composition to the patient, and introducing a cdtR mutant gene to a bacterium.

Disclosed are methods for treating and/or preventing C. difficile pathogenesis, the method comprising administering to a patient a C. difficile cell having a reduced cdtR protein expression relative to a wild type C. difficile. Disclosed are methods for treating and/or preventing C. difficile pathogenesis, the method comprising administering to a patient a C. difficile cell that expresses a mutant cdtR protein. Disclosed are methods for treating and/or preventing C. difficile pathogenesis, the method comprising administering to a patient a C. difficile cell that does not express a functional cdtR protein.

In certain aspects, the C. difficile strain comprises ST1-75 (which may be referred to as MSK-CD75), which was deposited under the Budapest Treaty with the American Type Culture Collection (ATCC) on May 3, 2023 (ATCC Deposit Number PTA-127589). In certain aspects, the C. difficile strain comprises ST1-35 (which may be referred to as MSK-CD35), which was deposited under the Budapest Treaty with the American Type Culture Collection (ATCC) on May 3, 2023 (ATCC Deposit Number PTA-127588). In certain aspects, the C. difficile cell has a mutant cdtR gene relative to wild type C. difficile. The wild type C. difficile may be the most abundant C. difficile strain in a normal population of C. difficile bacteria. The wild type C. difficile may be the C. difficile strain known to one of ordinary skill in the art as being the wild type C. difficile. The wild type C. difficile may be a virulent C. difficile. The wild type C. difficile may be a common, or the most common, strain of C. difficile found in one or more patients with colitis. In certain aspects, the mutant cdtR gene comprises a nonsense mutation, a single nucleotide variation, and/or a deletion. Also disclosed are mutant cdtR gene encoding for non-functional cdtR proteins and/or cdtR proteins with reduced function relative to wild type cdtR. The cdtR mutant may have a deletion, including the 69-base pair deletion of SEQ ID NO:9. The cdtR mutant may comprise SEQ ID NO:8. The cdtR mutant may have a deletion that is functionally and/or substantially equivalent to the 69-base pair deletion of SEQ ID NO:9.

Disclosed are C. difficile strains having a mutant cdtR gene and/or expressing or having a mutant cdtR protein. Also disclosed are C. difficile strains having reduced or absent cdtR protein levels compared to wild type C. difficile strains. Also disclosed are C. difficile strains that do not express functional cdtR protein. It is specifically contemplated that, in some aspects, a strain may not have a mutation that causes reduced expression of a cdtR gene. Also disclosed are engineered C. difficile cells. The engineered C. difficile cell may be engineered to comprise a cdtR mutant, including any cdtR mutant disclosed herein. The engineered C. difficile cell may be used in methods disclosed herein, including method where a C. difficile cells is administered to a patient. Certain aspects relate to an avirulent C. difficile cell, which may be an engineered C. difficile cell, such as an engineered C. difficile cell comprising a cdtR mutant. The avirulent C. difficile cell may be ST1-35. The avirulent cell may be ST1-75. The avirulent cell may be cdtRmut6.1. The avirulent cell may be cdtRmut8.1.

In certain aspects, at least one of the disclosed C. difficile strains are administered to a patient. Certain aspects also relate to compositions comprising at least one of the C. difficile cells disclosed herein. Such composition may be a therapeutic composition and may comprise more than one C. difficile cell strain and/or one or more additional therapies. In certain aspects, one or more additional therapies are administered to the patient. The additional therapies may be another C. difficile strain, an antibiotic, or a therapy for colitis.

Also disclosed are methods for analyzing virulence of a bacterial strain, including a C. difficile strain. The bacterial strain may be a bacterial strain present in an infection. Also disclosed are methods of treating a bacterial infection where a cdtR gene has been identified in the bacterial infection. The infection may be in the colon of a patient. The bacterial strain may be analyzed from a biological sample taken from a patient infected with the bacterial strain. Analyzing the virulence may comprise identifying a cdtR gene present in the bacterial strain.

The identified cdtR gene may be the wild type cdtR gene, including the C. difficile wild type cdtR gene, such as the cdtR present in a virulent bacterial strain. The bacterial strain may be R20291. The bacterial strain may be VPI10463. In certain aspects, where the wild type cdtR gene is identified, the bacterial strain may be considered virulent. In certain aspects where the wild type cdtR gene is not identified, the bacterial strain may be considered avirulent.

The identified cdtR gene may be a mutant cdtR gene, including any mutant cdtR gene disclosed herein.

In certain aspects, the bacterial strain being considered virulent or avirulent can determine a treatment regimen for the patient. For example, if the bacterial strain is considered virulent, a treatment regimen may comprises at least one of the C. difficile strains comprising a cdtR mutant and/or one or more antibiotics. Additionally for example, if the bacterial strain is considered avirulent, a treatment regimen may comprise an antibiotic.

Also disclosed are methods of reducing the virulence of a bacterium by introducing a cdtR mutant to the bacterium. The cdtR mutant may be any cdtR mutant disclosed herein. The bacterium may be a C. difficile bacterium, including R20291. The cdtR mutant may be introduced to the bacterium in vitro. The cdtR mutant may be introduced to the bacterium in vivo, including where the bacterium is infecting a patient. The cdtR mutant gene may be introduced by infecting the bacterium with a bacteriophage that encodes the cdtR mutant gene. The cdtR mutant gene may be introduced by bacterial transformation. The cdtR mutant may be a cdtR knock out mutant. The cdtR mutant may be introduced by genetic engineering, such as CRISPR-mediated genome edits.

Also disclosed are bacteriophages encoding a mutant cdtR gene, including any mutant cdtR gene disclosed herein. The bacteriophages may be derived from prophages, including any prophage disclosed herein, such as phiCD75-2 and/or phiCD75-3.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one.” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise.” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C. Clinical ST1 C. difficile isolates demonstrated variable virulence in mice treated with antibiotics. (A) Plot of the UMAP analysis of the presence or absence of unique coding sequences (annotated proteins or un-annotated protein clusters) across top 10 STs of C. difficile strains in Patric. (B) Mouse experiment schematic: wildtype C57BL/6 mice were treated with metronidazole, vancomycin, and neomycin (MNV. 0.25 g/L for each) in drinking water for 3 days and followed by one intraperitoneal injection of clindamycin (200 μg/mouse) 2 days after antibiotic recess. Then, mice were inoculated with 200 C. difficile spores via oral gavage. Daily body weight and acute disease scores were monitored for 7 days post infection. (C) % Max weight loss to baseline were calculated using the lowest weights within 7 days post infection divided by day 0 weights. N (Number of mice per strain-infected group)=5-8 except for ST1-62 and ST1-68, which have 2 mice per group.

FIGS. 2A-2H. Two isolated clinical strains of C. difficile have no virulence in mice treated with antibiotics. Wildtype C57BL/6 mice (n=3-5 per group) were treated with metronidazole, vancomycin, and neomycin (MNV. 0.25 g/L for each) in drinking water for 3 days and followed by one intraperitoneal injection of clindamycin (200 μg/mouse) 2 days after antibiotic recess. Then, mice were inoculated with 200 C. difficile spores via oral gavage. Daily body weight and acute disease scores were monitored for 7 days post infection. (A, D) % Weight loss to baseline of mice infected with indicated strains. (B. E) Acute disease scores comprising weight loss, body temperature drop, diarrhea, morbidity of mice infected with indicated strains. (C, F) Fecal colony-forming units measured by plating on selective agar on indicated days. (G-H) Fecal toxins measured by CHO cell rounding assay 1 day post infection.

FIGS. 3A-3H. Avirulent C. difficile strain demonstrates no virulence in innate immune deficient mice and germ-free mice. (A-D) MyD88/mice (n=4 per group) were treated with MNV and clindamycin before orally administered with 200 spores of C. difficile strains. Daily body weight and acute disease scores were monitored for 7 days post infection. (A) % Weight loss to baseline of mice infected with indicated strains. (B) Acute disease scores comprising weight loss, body temperature drop, diarrhea, morbidity of mice infected with indicated strains. (C) Fecal colony-forming units measured by plating on selective agar 1 day post infection. (D) Fecal toxins measured by CHO cell rounding assay 1 day post infection. (E-H) Germ-free mice (n=3 to 5) orally administered with 200 spores of indicated C. difficile strains. Daily body weight and acute disease scores were monitored for 10 days post infection. (E) % Weight loss to baseline of mice infected with indicated strains. (F) Diarrhea scores of mice infected with indicated strains 2 days post infection. (G) Fecal colony-forming units measured by plating on selective agar at 6, 12, and 24 hours post infection. (H) Fecal toxins measured by CHO cell rounding assay at 6, 12, and 24 hours post infection. Statistical significance was calculated by Unpaired t-test. * p<0.05. ** p<0.01.

FIGS. 4A-4E. Prophages identified in avirulent C. difficile mildly impact virulence in mice treated with antibiotics. (A) Anvi'o plot displaying accessory genomes of ST1 isolates. Highlighted gene clusters in purple are unique to ST1-35 and ST1-75. Blue dashes (outmost layer) indicate phage-related genes by NCBI COG. (B) Schematic of mutant strains made using R20291 C. difficile strain. (C-E) Wildtype C57BL/6 mice (n=4 per group) were treated with MNV and clindamycin as preciously described. Then, mice were inoculated with 200 C. difficile spores via oral gavage. Daily body weight and acute disease scores were monitored for 7 days post infection. (C) % Weight loss to baseline of mice infected with indicated strains. (D) Acute disease scores comprising weight loss, body temperature drop, diarrhea, morbidity of mice infected with indicated strains. (E) Fecal colony-forming units measured by plating on selective agar 1 day post infection.

FIGS. 5A-5G. Binary toxin regulator cdtR contributes to C. difficile virulence in mice. (A) Deletion identified in ST1-35/75 and schematic of cdtR mutants generated using R20291 C. difficile strain. (B) Germ-free mice (n=3 per group) orally administered with 200 spores of indicated C. difficile strains. Binary toxin gene cdtB transcripts were measured by RT-qPCR on cecal contents harvested at 24 hours post infection. Transcripts were normalized to the adk and fold change is relative to ST1-75 condition. (C-E) Wildtype C57BL/6 mice (n=3 to 5 per group) were treated with MNV and clindamycin as preciously described. Then, mice were inoculated with 200 C. difficile spores via oral gavage. Daily body weight and acute disease scores were monitored for 7 days post infection. (C) % Weight loss to baseline of mice infected with indicated strains. (D) Acute disease scores comprising weight loss, body temperature drop, diarrhea, morbidity of mice infected with indicated strains. (E) Fecal toxins measured by CHO cell rounding assay on indicated days. (F-G) Germ-free mice (n=4) orally administered with 200 spores of indicated C. difficile strains. Daily body weight and were monitored for 5 days post infection. (F) % Weight loss to baseline of mice infected with indicated strains. (G) Diarrhea scores of mice infected with indicated strains 3 days post infection. Statistical significance was calculated by One-way ANOVA. * p<0.05. ** p<0.01.

FIGS. 6A-6B. CdtR regulates PaLoc toxins transcription in vivo. Germ-free mice (n=4) were orally administered with 200 spores of indicated C. difficile strains and cecal contents were harvested at 24 hours post infection. (A) Cecal toxins measured by CHO cell rounding assay. (B) Indicated gene transcripts were measured by RT-qPCR. Transcripts were all normalized to the adk and fold change is relative to ST1-75 for each of the genes. Statistical significance was calculated by Unpaired t-test. * p<0.05. ** p<0.01. **** p<0.0001.

FIG. 7. Binary toxin regulator cdtR is prevalent in clinical C. difficile isolates. Binary toxin cdtA, cdtB and cdtR from R20291 were used as query to BLAST against the assembled contigs. Hits with at least 85% identity and 85% coverage of the query were considered a valid match. Numbers of match in total and percentages are presented.

FIGS. 8A-8D. ST1 C. difficile strains are closely related. (A) Pathogenicity loci were extracted from whole-genome sequence and multiple sequence alignment was performed for all strains. Each dash indicates one single-nucleotide polymorphism. (B) Average nucleotide identity was calculated with pairs of ST1 isolates. (C) Survival curve of indicated strain over a 7-day time course post infection. (D) Fecal colony-forming units measured by plating on selective agar on 1 day post infection from FIG. 1C.

FIGS. 9A-9D. Avirulent C. difficile strain demonstrates no virulence in germ-free mice. Germ-free mice (n=4 per group) orally administered with 200 spores of indicated C. difficile strains. Daily body weight and acute disease scores were monitored for 6 days post infection. (A) % Weight loss to baseline of mice infected with indicated strains. (B) Diarrhea scores of mice infected with indicated strains on 3 days post infection. (C) Fecal colony-forming units measured by plating on selective agar 1 day post infection. (D) Fecal toxins measured by CHO cell rounding assay 1 day post infection. Statistical significance was calculated by Unpaired t-test. * p<0.05. ** p<0.01.

FIGS. 10A-10D. Unique prophages identified in two avirulent C. difficile strains. (A) Schematic of prophages identified in avirulent strains. Each arrow represents a coding sequence whose product is indicated, when available. Numbers refer to the CDS when no function could be assigned. Gene products are colored by functional groups; yellow=packaging; red=head morphogenesis proteins; blue=tail morphogenesis proteins; cyan=lysis module; green=gene regulation and DNA replication; orange=lysogeny; grey=other or unknown function. Maps were generated with Benchling and finalized in Inkscape v1.2. (B-D) Wildtype C57BL/6 mice (n=4 per group) were treated with MNV and clindamycin as preciously described. Then, mice were inoculated with 200 C. difficile spores via oral gavage. (B) Fecal toxins measured by CHO cell rounding assay 1 day post infection. (C) Fecal colony-forming units measured by plating on selective agar 7 days post infection. (D) Fecal toxins measured by CHO cell rounding assay 7 days post infection.

FIGS. 11A-11G. Binary toxin regulator cdtR does not impact C. difficile colonization in mice. (A) Schematic of cdtR mutants generated using R20291 C. difficile strain. (B-F) Wildtype C57BL/6 mice (n=3 to 5 per group) were treated with MNV and clindamycin as preciously described. Then, mice were inoculated with 200 C. difficile spores via oral gavage. Daily body weight and acute disease scores were monitored for 7 days post infection. (B) % Weight loss to baseline of mice infected with indicated strains. (C) Acute disease scores comprising weight loss, body temperature drop, diarrhea, morbidity of mice infected with indicated strains. (D, F) Fecal colony-forming units measured by plating on selective agar 1 day post infection (E) Fecal toxins measured by CHO cell rounding assay on indicated days. (G) Germ-free mice (n=4) orally administered with 200 spores of indicated C. difficile strains. Fecal colony-forming units measured by plating on selective agar 1 day post infection. Statistical significance was calculated by One-way ANOVA. * p<0.05.

FIGS. 12A-12E. CdtR regulates PaLoc toxins production. (A-B) Wildtype C57BL/6 mice (n=3-5 per group) were treated with MNV and clindamycin as preciously described. Then, mice were inoculated with 200 C. difficile spores via oral gavage. (A) Fecal toxins measured by CHO cell rounding assay 7 days post infection for ST1-35. (B) Fecal toxins measured by CHO cell rounding assay 14 days post infection for ST1-75. (C-E) Germ-free mice (n=3-4) orally administered with 200 spores of indicated C. difficile strains and cecal contents were harvested at 24 hours post infection. (C) Cecal CFU measured by plating on selective agar. (D) Cecal toxin as in FIG. 5A was normalized to CFU. (E) PaLoc and CdtLoc transcripts were measured by RT-qPCR. Transcripts were all normalized to the adk and fold change is relative to CdtRmut_6.1 for each of the genes. Statistical significance was calculated by Unpaired t-test, * p<0.05, ** p<0.01.

FIGS. 13A-13C. ST1-75/35 harbors unique mutations in cdtR. (A) cdtR hits without starting position at the beginning of C. difficile contigs were chosen to further examine their nucleotide differences in cdtR gene to R20291 and ST1-75. Five out of 491 ST1 isolates (from both strain collection databases) were found to have nucleotide variants. (B) Phylogenetic tree build based on core genome snps of ST1 isolates against R20291. (C) Timeline of hospital stay and spatial overlap between the two patients harboring ST1-75 and ST1-35.

FIGS. 14A-14E. Avirulent C. difficile strain provides protection against virulent infection in mice. (A) Schematic showing experimental procedure (B) % Weight loss to baseline of mice infected with indicated strains. (C) Acute disease scores comprising weight loss, body temperature drop, diarrhea, morbidity of mice infected with indicated strains. (D) Fecal colony-forming units measured by plating on selective agar on 1 day post infection. (E) Wildtype cdtR copies in feces from infected mice were measured by qPCR on 1 day post infection. Statistical significance was calculated by One-way ANOVA. **** p<0.0001.

FIGS. 15A-15B. Challenge of virulent infection with ST1-75 can replace R20291. (A) % Weight loss to baseline of mice infected with indicated strains. 1:1 ratio of ST1-75 and R20291 co-administered on time 0 (triangle), R20291 administered at time 0 followed by 200 ST1-75 spores 6 hrs later (upside-down triangle), R20291 administered at time 0 followed by 104 ST1-75 spores 6 hrs later (diamond), R20291 administered at time 0 followed by 104 ST1-75 spores 24 hrs later (circle), R20291 administered at time 0 followed by 105 vegetative ST1-75 24 hrs later (square). (B) Wildtype cdtR copies in feces from infected mice were measured by qPCR on 1 day and 7 days post infection. Statistical significance was calculated by One-way ANOVA. * p<0.05. ** p<0.01. **** p<0.0001. Y-axis indicates cdtR region, proxy for R20291 in fecal sample; by day 7, R20291 signal is decreased in the following conditions: R20291 administered at time 0 followed by 200 ST1-75 spores 6 hrs later, R20291 administered at time 0 followed by 104 ST1-75 spores 6 hrs later, 1:1 ratio of ST1-75 and R20291 administered on time 0, however, R20291 signal is not decreased when administered after 24 hrs.

FIGS. 16A-16C. Co-infection with cdtRmut is not protective against symptoms (weight, acute disease score), yet replaces WT strains. (A) Fecal colony-forming units measured by plating on selective agar on 1 day post infection. (B) % Weight loss to baseline of mice infected with indicated strains: mice administered cdtRmut (circle), R20291 (square), or 1:1 ratio of cdtRmut and R20291 (hexagon). (C) Wildtype cdtR copies in feces from infected mice were measured by qPCR on 1 day. 5 days and 7 days post infection. Statistical significance was calculated by One-way ANOVA. **** p<0.0001. Y-axis indicates cdtR region, proxy for R20291 in fecal sample; by day 8, R20291 signal is decreased in the following conditions: 1:1 ratio of cdtRmut and R20291 administered on time 0.

FIGS. 17A-17B. cdtRmut 6h challenge also replaces R20291. (A) % Weight loss to baseline of mice infected with indicated strains: mice administered cdtRmut (circle), R20291 (square), R20291 administered at time 0 followed by 200 cdtRmut spores 6 hrs later (R20291>CdtRmut, diamond) or cdtRmut administered at time 0 followed by 200 R20291 spores 6 hrs later (R20291>CdtRmut, diamond). (B) Wildtype cdtR copies in feces from infected mice were measured by qPCR on 1 day, 2 days and 21 days post infection. Y-axis indicates cdtR region, proxy for R20291 in fecal sample; by day 3 to day 21, R20291 signal is decreased in the following conditions: R20291 administered at time 0 followed by 200 cdtRmut spores 6 hrs later (R20291>CdtRmut) and cdtRmut administered at time 0 followed by 200 R20291 spores 6 hrs later (R20291>CdtRmut).

FIGS. 18A-18D. CdtRmut replacing virulent strain leads to protection upon transmission. (A) Schematic showing experimental setup: C. difficile infected mice is co-housed with antibiotics-treated mice (that are susceptible to transmission). (B) Fecal colony-forming units measured by plating on selective agar on 1 day post infection. (C) % Weight loss to baseline of mice co-housed with infected mice priorly with indicated strains: mice administered cdtRmut (circle). R20291 (square). 1:1 ratio of cdtRmut and R20291 (hexagon). (D) Wildtype cdtR copies in feces from infected mice were measured by qPCR on 1 day post infection. Y-axis indicates cdtR region, proxy for R20291 in fecal sample; X-axis indicate the body weight of transmitted mice. Less body weight loss correlates with less wildtype strain transmitted.

FIGS. 19A-19D. ST1-75 protection is microbiome independent. CdtRmut replacing virulent strain leads to protection upon transmission. (A) % Weight loss to baseline of mice infected with indicated strains: mice administered ST1-75 (circle). R20291 (square). 1:1 ratio of ST1-75 and R20291 (upside-down triangle). (B) Diarrhea scores of mice infected with indicated strains 2 days post infection. (C) Fecal colony-forming units measured by plating on selective agar on 1 day post infection. (D) Wildtype cdtR copies in feces from infected mice were measured by qPCR on 1 day post infection.

FIGS. 20A-20C. Avirulent C. difficile provides long-term protection via colonization resistance. (A) Schematic showing experimental procedure (B) % Weight loss to baseline of mice infected with indicated strains. Mice pre-treated with H₂O (black circle), mice pre-treated with H₂O and challenged with R20291 (H2O>R20291, square), mice pre-inoculated with ST1-75 (green circle), mice pre-inoculated with ST1-75 and challenged with R20291 (ST1-75>R20291, upside-down triangle). (C) Fecal colony-forming units measured by plating on selective agar on indicated days post infection. ST1-75 survived antibiotics challenge and bounced back to high levels to provide colonization resistance against virulent C. difficile R20291.

FIGS. 21A-21D. Figure S3 Avirulent C. difficile provides long-term protection to prevent virulent recurrence. (A) % Weight loss to baseline of mice infected with indicated strains. Mice pre-treated with H₂O and challenged with R20291 (H2O>R20291, square), mice pre-inoculated with cdtRmut (circle), mice pre-inoculated with cdtRmut and challenged with R20291 (cdtRmut >R20291, hexagon). (B) Fecal colony-forming units of cdtRmut measured by plating on selective agar on indicated days post infection. CdtRmut survived antibiotics challenge to provide colonization resistance against virulent C. difficile R20291. (C) % Weight loss to baseline of mice infected with indicated strains. Mice pre-inoculated with ST1-75 and challenged with R20291 (ST1-75>R20291, circle), mice pre-inoculated with R20291 and challenged with R20291 (R20291>R20291, square), mice pre-inoculated with 1:1 ratio of ST1-75 and R20291, and challenged with R20291 (ST1-75: R20291>R20291, upside-down triangle). (D) Fecal colony-forming units of resulting C. difficile from each condition in S3C measured by plating on selective agar on indicated days post infection. Antibiotics treatment induce virulent recurrence of C. difficile infection in mice infected with R20291 previously. However, introducing ST1-75 prior (in the case of ST1-75>R20291 and ST1-75: R20291>R20291) protected mice form virulent recurrence.

### DETAILED DESCRIPTION OF THE INVENTION

Clostridioides difficile produces toxins that damage the colonic epithelium and causes colitis. Variation in disease severity is poorly understood and has been attributed to host factors and virulence differences between C. difficile strains. At least 23 epidemic ST1 C. difficile clinical isolates are disclosed herein. In some aspects, the isolate comprises Certain isolates encode a complete Ted pathogenicity locus and achieved similar colonization densities. However, disease severity may vary from lethal to avirulent infections, in certain aspects. In certain aspects, the bacteria comprise a 69-bp deletion in the cdtR gene, which encodes a response regulator for binary toxin expression. Deleting the 69-bp sequence in virulent bacteria, such as the R20291 strain, may render it avirulent, including in mice with reduced toxin gene transcription. Certain aspects herein demonstrate that a natural deletion within cdtR can attenuate virulence in the epidemic ST1 C. difficile strain without reducing colonization and persistence. Distinguishing strains on the basis of cdtR may enhance the specificity of diagnostic tests for C. difficile colitis.

Certain aspects herein relate to the use of an antibiotic-treated mouse model of C. difficile infection to test a panel of PaLoc- and CdtLoc-encoding ST1 C. difficile clinical isolates to quantify disease severity.²⁰ Clinical C. difficile isolates with identical PaLoc are disclosed to cause a range of disease severities, with at least two isolates disclosed to cause no detectable disease in antibiotic-treated wild type, germ-free mice or Myd88-defecient mice. Also disclosed are deletions in the cdtR gene, including a 69-bp deletion in the cdtR gene of the two avirulent isolates, which encodes a LytTR family response regulator that regulates CDT expression. The 69-bp deletion in the cdtR leads to reduced CDT toxin and PaLoc gene expression, resulting in loss of virulence and confirming previous studies implicating CdtR as regulator of CDT and Ted toxins expression.²¹ Studies herein are the first to describe virulence diversity within a single strain type and demonstrates the critical role of CdtR for ST1 C. difficile virulence.

### I. Proteins

As used herein, a “protein” or “polypeptide” refers to a molecule comprising at least five amino acid residues. As used herein, the term “wild type” refers to the most prevalent allele of a gene in a certain species. In some aspects, a wild type cdtR encodes a fully functional protein that positively regulate downstream CDT toxin expression and TcdA and TcdB expression. A “variant” or a “mutant” refers to an allele or gene sequence encoding a protein or polypeptide whose chemical structure, particularly its amino acid sequence, is altered with respect to the wild-type protein or polypeptide. In some embodiments, a mutant/variant protein or polypeptide has at least one modified activity or function (recognizing that proteins or polypeptides may have multiple activities or functions). It is specifically contemplated that a mutant/variant protein or polypeptide may be altered with respect to one activity or function yet retain a wild-type activity or function in other respects.

In particular embodiments, there are isolated nucleic acid segments, recombinant vectors, and/or phages incorporating nucleic acid sequences that encode a cdtR mutant polypeptide. The term “recombinant” may be used in conjunction with a polypeptide or the name of a specific polypeptide, and this generally refers to a polypeptide produced from a nucleic acid molecule that has been manipulated in vitro or that is a replication product of such a molecule.

It is contemplated that a cdtR wild type protein may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The cdtR proteins or polynucleotides encoding cdtR may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOs1-9.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOS: 1-9.

In some embodiments, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOS: 1-9.

In some embodiments, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOS: 1-9 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS: 1-9.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS: 1-9 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS: 1-9.

The nucleotide as well as the protein, polypeptide, and peptide sequences for cdtR have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, protein, nucleic acid per mL. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/mL or more (or any range derivable therein).

### 1. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 1, 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of non-contiguous or contiguous amino acids can be deleted, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### 2. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalaninc (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within +2 is included. In some aspects of the present disclosure, those that are within +1 are included, and in other aspects of the present disclosure, those within +0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within +2 are included, in other embodiments, those which are within +1 are included, and in still other embodiments, those within +0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the disclosure, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### II. Formulations and Culture of the Cells

In particular embodiments, the cells of the disclosure, including a C. difficile cell such as a C. difficile cell comprising any of the cdtR genes disclosed herein, may be specifically formulated and/or they may be cultured in a particular medium. In certain aspects, C. difficile ST1-35 is formulated and/or cultured. In certain aspects, C. difficile ST1-75 is formulated and/or cultured. In certain aspects, C. difficile cdtRmut6.1 is formulated and/or cultured. In certain aspects, C. difficile cdtRmut8.1 is formulated and/or cultured. The cells may be formulated in such a manner as to be suitable for delivery to a recipient without deleterious effects, including after sufficient culturing. Also disclosed are the C. difficile cells for use in methods described herein.

In some aspects, bacteria, including C. difficile, are isolated and/or purified from a source. The bacteria may be isolated and/or purified from any suitable source. In some aspects, the source is a human sample. In some aspects, the source is a non-human sample, such as a rodent sample. Bacteria, including C. difficile, may be isolated and/or purified using any method known in the art. The isolated and/or purified bacteria may then be formulated into therapeutic compositions, including the therapeutic compositions described herein. Before or after being isolated and/or purified, the bacteria may be characterized, including by sequencing to determine the composition and identity of the isolated and/or purified bacteria as C. difficile or any C. difficile strain disclosed herein. The bacteria may be characterized by 16S rRNA or 23S rRNA sequencing.

In certain aspects, the methods described herein of treating a patient and methods comprising administering C. difficile, including any C. difficile strain disclosed herein, to a patient further comprise isolating and/or purifying the C. difficile prior to the treatment and/or administration. The purified and/or isolated C. difficile may be used in the methods described herein.

In some embodiments, bacteria may be cultured for at least between about 1 day and about 40 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art, including any LB medium. Compositions of the above basal media are generally known in the art, and it is within the skill of one in the art to modify or modulate concentrations of media and/or media supplements as necessary for the cells cultured. Any medium capable of supporting cells in vitro may be used to culture the cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular embodiments, cells are cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

Suitable methods for nucleic acid delivery to effect expression of compositions are anticipated to include virtually any method by which a nucleic acid (e.g., DNA, including viral and nonviral vectors) can be introduced into a cell or an organism, as described herein or as would be known to one of ordinary skill in the art. Such methods include, but are not limited to, direct delivery of DNA such as by injection or transformation.

Also disclosed are culture systems capable of producing bacteriophage, including any bacteriophage disclosed herein such as bacteriophage encoding a mutant cdtR gene. The bacteriophage may be derived from a prophage, including from phiCD75-2 and/or phiCD75-3. The bacteriophage may be isolated and/or purified from a source. The bacteriophage may be formulated for use in a therapeutic composition, including any of the therapeutic compositions disclosed herein. The bacteriophage, including when formulated in a therapeutic composition, may be administered to a patient.

Sequences for aspects disclosed herein include

Whole genome sequence for C. difficile ST1-35 (SEQ ID NO:1)

Whole genome sequence for C. difficile ST1-75 (SEQ ID NO:2)

Whole genome sequence for C. difficile cdtRmut6.1 (SEQ ID NO:3)

Whole genome sequence for C. difficile cdtRmut8.1 (SEQ ID NO:4)

Whole genome sequence for phiCD75-2 (SEQ ID NO:5)

Whole genome sequence for phiCD75-3 (SEQ ID NO:6)

Wild-type C. difficile cdtR gene
(SEQ ID NO: 7)
GTGGATATATTAATTTTTGATAATGATGTTTGTTTTGGAATAAAA
CTAAAAGAAAAAATAAATAATATTTTAATAAAAGAAGGTTTTGAT
AACGATGTTATAAGATTATATTATAATGCAAATTTGTTATTAAAA
GAACTTACTGAAAAAAATAAAGTAAAAATATATTTTATAGTTGTA
GATGCAAAATACAAGATAAGTAATGAATTATGTGATGGATTATGG
ATAGCACAAAAAATTAGAGAATCAGATTATATTAGTCCAATCATA
TTTCTTACAAATCATATAGAAATGATTTTAGGGATCTTCGATTAT
AGGTTAGAAGTTATGGACTTTATATTAAAACATGATATGGAAATT
GCTGAAAGTAAAATAAAAGCTTGTATTAAAATTGCTCATAAAAGA
TACGTTAAAGAAAAAAATTATCGCTCTAATTTTTTTACTATCTAC
TCAGATTCCTCACTATGGAAAATTTCATTTGATGAAGTTATATAT
TTTGAAACAAGCGCTATTCCACATAAAATAAAATTAGTGACTACT
TCAAGAATATTTGAATTCTATAAAAGTTTAAGGTCTTTATCAGAT
TTAGATGCATGCTTTATTCGTGTACATAAATCTTTTGTAGTTAAT
AAATATCATATTGTTTCTCTCGACCTAAAGAAAAATAATATTAAA
ATGAGTAATGGCCATATATGTCGTATATCTAATACATATAGAAAT
ATTTTAAAGAACATTATTAAAACATAA
Avirulent C. difficile cdtR gene containing
the 69-base pair deletion
(SEQ ID NO: 8)
GTGGATATATTAATTTTTGATAACGATGTTATAAGATTATATTAT
AATGCAAATTTGTTATTAAAAGAACTTACTGAAAAAAATAAAGTA
AAAATATATTTTATAGTTGTAGATGCAAAATACAAGATAAGTAAT
GAATTATGTGATGGATTATGGATAGCACAAAAAATTAGAGAATCA
GATTATATTAGTCCAATCATATTTCTTACAAATCATATAGAAATG
ATTTTAGGGATCTTCGATTATAGGTTAGAAGTTATGGACTTTATA
TTAAAACATGATATGGAAATTGCTGAAAGTAAAATAAAAGCTTGT
ATTAAAATTGCTCATAAAAGATACGTTAAAGAAAAAAATTATCGC
TCTAATTTTTTTACTATCTACTCAGATTCCTCACTATGGAAAATT
TCATTTGATGAAGTTATATATTTTGAAACAAGCGCTATTCCACAT
AAAATAAAATTAGTGACTACTTCAAGAATATTTGAATTCTATAAA
AGTTTAAGGTCTTTATCAGATTTAGATGCATGCTTTATTCGTGTA
CATAAATCTTTTGTAGTTAATAAATATCATATTGTTTCTCTCGAC
CTAAAGAAAAATAATATTAAAATGAGTAATGGCCATATATGTCGT
ATATCTAATACATATAGAAATATTTTAAAGAACATTATTAAAACA
TAA
69-base pair deletion from the C. difficile
cdtR gene causing avirulence
(SEQ ID NO: 9)
TTTGATAATGATGTTTGTTTTGGAATAAAACTAAAAGAAAAAATA
AATAATATTTTAATAAAAGAAGGT

### III. Gene Editing Systems

Certain aspects of the present disclosure relate to methods and compositions for gene editing (also “genetic engineering”), useful in the generation of one or more genetic modifications in a cell, including genetic modifications to a cdtR gene in the cell. As used herein, a “genetic modification,” describes a region of a genome of a cell that has been altered from its native (i.e., endogenous) sequence. A genetic modification may be developed via artificial editing of a gene or other genetic material. In some embodiments, a genetic modification is a mutation of a gene. In some embodiments, a mutation is an insertion, a deletion, a point mutation, a frameshift mutation, or a nonsense mutation. One or more of these mutations may be excluded from embodiments of the disclosure. In some embodiments, a mutation prevents expression of a gene (i.e., is a knockout mutation). In some embodiments, a mutation causes production of a mutant form of a protein. In some embodiments, a genetic modification of the disclosure is a mutation of a cdtR gene.

Certain embodiments of the disclosure are directed to the use of gene editing techniques to generate a knockout mutation in a gene in a population of cells. The disclosed techniques may eliminate expression of the gene in some or all of the cells in the population. In some embodiments, expression of the gene is not detectable in the population of cells (e.g., measured by mRNA and/or protein expression). In some embodiments, expression of the gene is substantially decreased in the population of cells compared with cells not having the knockout mutation. In some embodiments, expression of the gene (e.g., measured by mRNA and/or protein expression) is decreased by at least, at most, or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, 99.9%, or any range or value derivable therein. In some embodiments, expression of the gene is decreased by at least 80%, 90%, 95%, or 99%. In some embodiments, expression of the gene is decreased by at least 90%.

Various methods and systems for gene editing are known in the art and include, for example, zinc finger nuclease (ZFN)-based gene editing, transcription activator-like effector nuclease (TALEN)-based gene editing, and CRISPR/Cas-based gene editing. In some embodiments, methods of the present disclosure comprise CRISPR/Cas-based gene editing, which comprises the use of components of a CRISPR system, for example a guide RNA (gRNA) and a Cas nuclease.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. A Cas nuclease and a gRNA can be introduced into the cell indirectly via introduction of one or more nucleic acids (e.g., vectors) encoding for the Cas nuclease and/or the gRNA. A Cas nuclease and a gRNA can be introduced into the cell directly by introduction of a Cas nuclease protein and a gRNA molecule. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA may be targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% sequence complementarity along the length of the tracr mate sequence when optimally aligned.

One or more vectors driving expression of one or more elements of a CRISPR system can be introduced into a cell such that expression of the elements of the CRISPR system direct formation of a CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding a Cas protein (also “Cas nuclease”). Non-limiting examples of Cas proteins include Cas1, Cas1B, Cas2, Cas3, Cas4, Cas5, Cas6, Cas7, Cas8, Cas9 (also known as Csn1 and Csx12), Cas10, Cas12a (Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The Cas nuclease can be Cas9 (e.g., from S. pyogenes or S. pneumonia). The Cas nuclease can be Cas12a. The Cas nuclease can direct cleavage of one or both strands at the location of a target sequence, such as within the target sequence and/or within the complement of the target sequence. The vector can encode a Cas nuclease that is mutated with respect to a corresponding wild-type enzyme such that the mutated Cas nuclease lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is or is more than 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

### IV. Administration of Therapeutic Compositions

Certain aspects concern the administration of therapies and therapeutic compositions, including any therapeutic composition described herein that includes C. difficile and/or a bacteriophage. The therapies may be administered in any suitable manner known in the art. The therapy provided herein may comprise administration of a combination of therapeutic composition, such as a first composition and a second composition. In some aspects, the first composition comprises any C. difficile comprising composition described herein. In some aspects, the second composition comprises an additional therapeutic agent, including any additional therapeutic agent described herein such as an antibiotic. The first and second compositions may be administered sequentially (at different times) or simultaneously (at the same time). In some aspects, the first and second compositions are administered as separate compositions. In some aspects, the first and second compositions are administered as the same composition.

In some aspects, the first therapeutic composition and the second therapeutic composition are administered substantially simultaneously. In some aspects, the first therapeutic composition and the second therapeutic composition are administered sequentially. In some aspects, the first therapeutic composition, the second therapeutic composition, and a third therapy, which may be a different additional therapy, are administered sequentially or simultaneously. In some aspects, the first and second therapeutic compositions are administered concurrently and the third therapy is administered sequentially, before and/or after, with the first and second therapeutic compositions. In some aspects, the first therapeutic composition is administered before administering the second therapeutic composition. In some aspects, the first therapeutic composition is administered after administering the second therapeutic composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed. In some aspects, the first composition, which may be a C. difficile composition, and optionally the second composition are administered prophylactically.

The therapeutic agents of the disclosure may be administered by the same route of administration or by different routes of administration. In some aspects, the therapeutic composition is administered intracolonically, intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. In some aspects, the composition is administered via gavage. The appropriate dosage may be determined based on the type of disease to be treated, severity and course of the disease, the clinical condition of the patient, the patient's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, a single dose of the first therapeutic composition, which comprises C. difficile, is administered. In some aspects, multiple doses of the first therapeutic composition are administered. In some aspects, the first therapeutic composition is administered at a dose of between 1×10³ to 9×10⁸ CFUs or spores, or any range derivable therein. In some aspects, the first therapeutic composition is administered at a dose of at least, at most, or about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, or any range derivable therein, CFUs or spores of the bacteria.

The quantity to be administered, both according to number of treatments and unit dose, depends on the treatment effect desired. An effective dose is understood to refer to an amount necessary to achieve a particular effect.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more (and any range derivable therein) administrations. The administrations can be at 1, 2, 3, 4, 5, 6, 7, 8, to 5, 6, 7, 8, 9, 10, 11, 12, 13, 14 day, week, or month intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

Administration of the compositions will typically be via any common route. This includes, but is not limited to oral, suppository, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

The desired dose of the composition of the present disclosure may be presented in multiple (e.g., two, three, four, five, six, or more) sub-doses administered at appropriate intervals throughout the day.

Upon formulation, solutions will be administered in a manner compatible with the dosage formulation and in such amount as is therapeutically or prophylactically effective. The formulations are easily administered in a variety of dosage forms, such as the type of injectable solutions described above.

### V. Methods of Treatment

Provided herein are methods for treating or delaying progression of certain diseases or disorders, such as intestinal diseases including colitis, by administration of therapeutic compositions, such as compositions comprising bacteria, including C. difficile and/or a bacteriophage, to a patient. The intestinal disorder may comprise intestinal inflammation including damage to the colonic epithelium. In some aspects, the C. difficile treatment results in a sustained response, such as a sustained microbiome, in the patient after cessation of the treatment.

The term “treatment” or “treating” means any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. In some aspects, the treatment may exclude prevention of the disease.

In certain aspects, the patient administered a therapeutic composition is identified as having, or is at risk of having, a disease, or disorder, including an intestinal disorder such as colitis. The colitis may be caused by a C. difficile infection. In certain aspects, the administration of a therapeutic composition to a patient alters the microbiome, including altering the bacteria in the microbiome, and/or microbiome environment in the patient. In some aspects, the administration of the therapeutic composition decreases the amount of at least one bacterial strain, bacteria, and/or genus of bacteria. In some aspects, the administration of the therapeutic composition decreases the amount of a wild-type C. difficile. In some aspects, the administration of the therapeutic composition alters the microbiome by altering the expression of at least one gene expressed in bacteria of the microbiome. The gene(s) may comprise a gene regulated by a cdtR protein. In some aspects, the administration of the therapeutic composition alters the regulation, production, and/or secretion of bacterial toxins.

### VI. Methods of Determining Bacterial Compositions

In some aspects, the methods relate to determining bacterial compositions present in a sample from a patient. In some aspects, obtaining a microbiome profile comprises the steps of or the ordered steps of: i) obtaining a sample obtained from a patient, ii) isolating one or more bacterial species from the sample, iii) isolating one or more nucleic acids from at least one bacterial species, and iv) analyzing the isolated nucleic acids. Analyzing the nucleic acids can comprise any method to determine the mutation status of a gene, including a cdtR gene. Analyzing the nucleic acid can comprise analyzing whether a gene is mutated, including whether a cdtR gene is mutated. Analyzing the nucleic acid can comprise PCR, sequencing, restriction enzyme analysis, or other methods to determine mutations. When performing the methods necessitating genotyping, any genotyping assay can be used.

In some aspects, determining bacterial compositions present in a sample of a patient is used to monitor the need of administering the therapeutic compositions described herein to the patient. In certain aspects, obtaining the microbiome profile of a patient is used to monitor the efficacy of the therapeutic compositions administered to the patient, including monitoring the concentration of C. difficile in the microbiome profile. In certain aspects, the patient is or is not administered a therapeutic composition based on the mutation status of a cdtR gene present in an infection in the patient. Methods for determining mutation status may include one or more microbiology methods such as sequencing, next generation sequencing, western blotting, comparative genomic hybridization, PCR, ELISA, etc.

Certain aspects relate to methods of prognosing the effectiveness of a therapy in a patient. In some aspects, the method prognoses the effectiveness of a bacterial and/or antibiotic therapy in a patient. The patient may have received, or will receive, an antibiotic. In certain aspects, the effectiveness of the therapy is determined by obtaining a microbiome profile from the patient.

### VII. Kits

Certain aspects of the disclosure also encompass kits for performing the methods of the disclosure. Such kits can be prepared from readily available materials and reagents. For example, such kits can comprise any one or more of the following materials: enzymes, reaction tubes, buffers, detergent, primers, probes, antibodies.

In a particular aspect, these kits may comprise a plurality of agents for assessing or identifying microorganisms, wherein the kit is housed in a container. The kits may further comprise instructions for using the kit for assessing sequences, means for converting and/or analyzing sequence data to generate prognosis.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

Further kit aspects relate to kits comprising the therapeutic compositions of the disclosure. The kits may be useful in the treatment methods of the disclosure and comprise instructions for use.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

A. Clinical ST1 C. difficile Isolates Demonstrate Variable Severities in Mice.

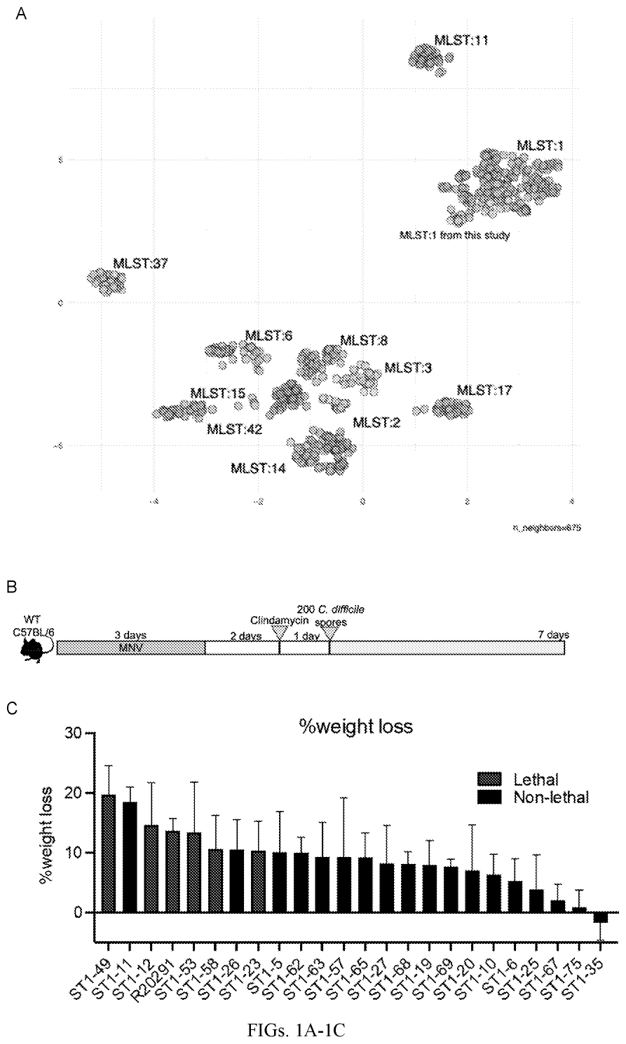

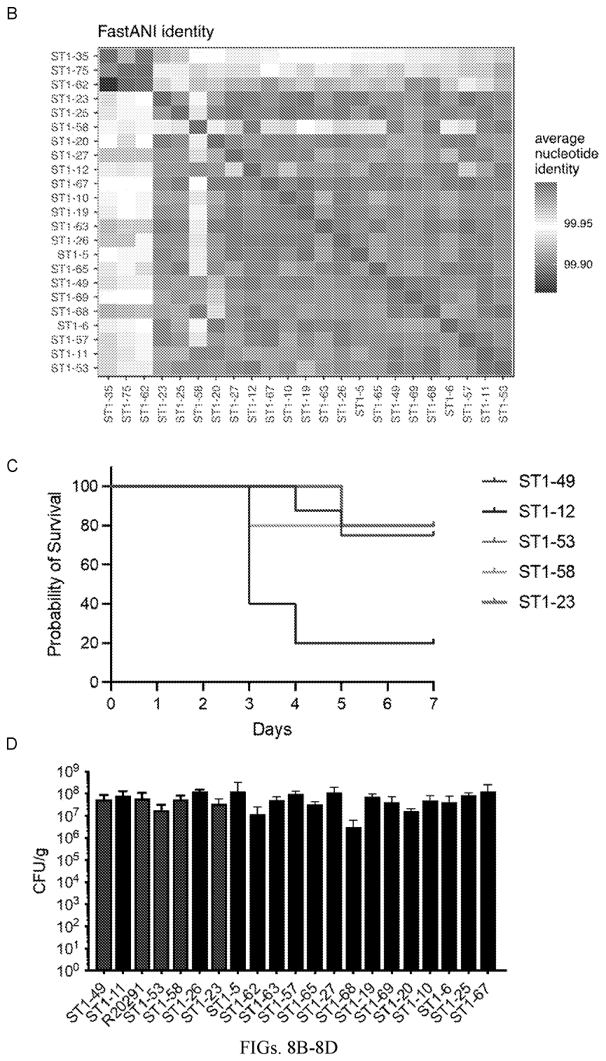

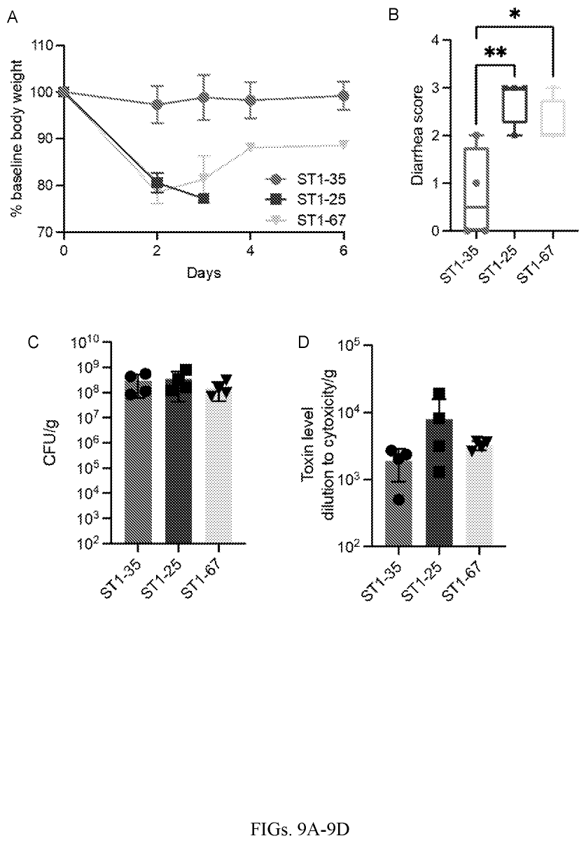

The inventors focus on a group of 23 C. difficile isolates belonging to ribotype 027 epidemic strains (here referred to as ST1) isolated from patients with diarrhea during a molecular surveillance program at Memorial Sloan Kettering Cancer Center 2013-2017.²² Whole-genome Illumina sequencing of these isolates allows a comparison between them and public collections. The inventors plotted a UMAP analysis of the presence or absence of unique coding sequences (annotated proteins or un-annotated protein clusters) across top 10 STs of C. difficile strains in Patric (date: Feb. 10, 2021).²³ Different STs cluster individually and the ST1 isolates overlap with other ST1 C. difficile included in the analysis, confirming their strain type (FIG. 1A). These 23 isolates demonstrate high genome-wide similarity by average nucleotide identity (ANI) score above 99.8% and encode identical Pathogenicity Locus (PaLoc) sequences (FIG. 8A-8B). To study if close-related C. difficile isolates may have variable virulence, mice treated with antibiotics (metronidazole, neomycin, vancomycin in drinking water with clindamycin intraperitoneal injection) were orally infected with each of these isolates at a dose of 200 spores and C. difficile pathogenicity was monitored throughout a 7-day-timecourse (FIG. 1B). Mice infected with different ST1 isolates displayed a spectrum of disease severity, including variable weight loss and mortality (FIG. 1C and FIG. 8C). The widely used ST1 lab strain R20291 was included in parallel for virulence comparison. Within the ST1 collection, 5 isolates resulted in mortality in mice. A few isolates caused more severe weight loss than R20291, including ST1-49, ST1-11 and ST1-12, while most ST1 isolates caused moderate and non-lethal infections. Two isolates, ST1-75 and ST1-35 demonstrated no impact on mouse body weights. No apparent colonization deficiency was observed in any of these isolates (FIG. 8D). The variable pathogenicity induced by a group of ST1 isolates with identical PaLoc suggested additional regulatory mechanisms of C. difficile virulence. Therefore, the inventors sought to examine other genomic factors that are responsible for attenuated virulence of C. difficile isolates ST1-75 and ST1-35.

B. Two ST1 C. difficile Isolates Demonstrate Avirulent Phenotype

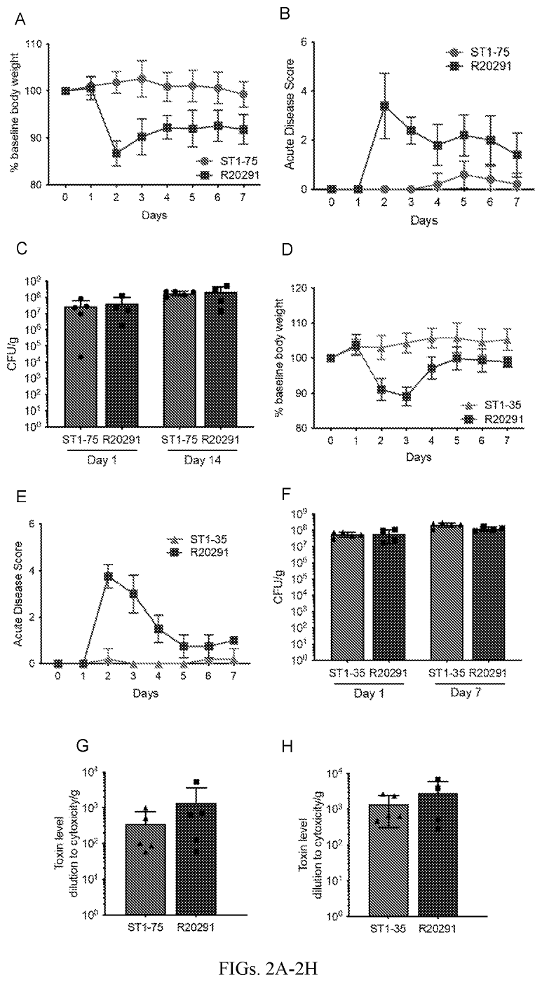

Among the C. difficile isolates that the inventors examined using antibiotic-treated mice, two isolates, ST1-75 and ST1-35, caught the inventors' attention due to their strikingly attenuated phenotypes (FIG. 1B). Almost no weight loss was observed throughout the 7-day-timecourse, and low acute disease scores were displayed in mice infected with ST1-75 or ST1-35, in comparison to mice infected with R20291 (FIGS. 2A-2B and 2D-2E). This avirulent phenotype was not due to colonization deficiency of ST1-75 or ST1-35, as the fecal CFU recovered from the mice infected with these two isolates were comparable to R20291-infected mice on both early and late days post-infection (FIGS. 2C and 2F). Fecal levels of TcdA and TcdB were also measured, and similar levels were seen in the feces at day +1 post-infection from mice infected with ST1-75 and ST1-35, compared to R20291 (FIG. 2G-2H).

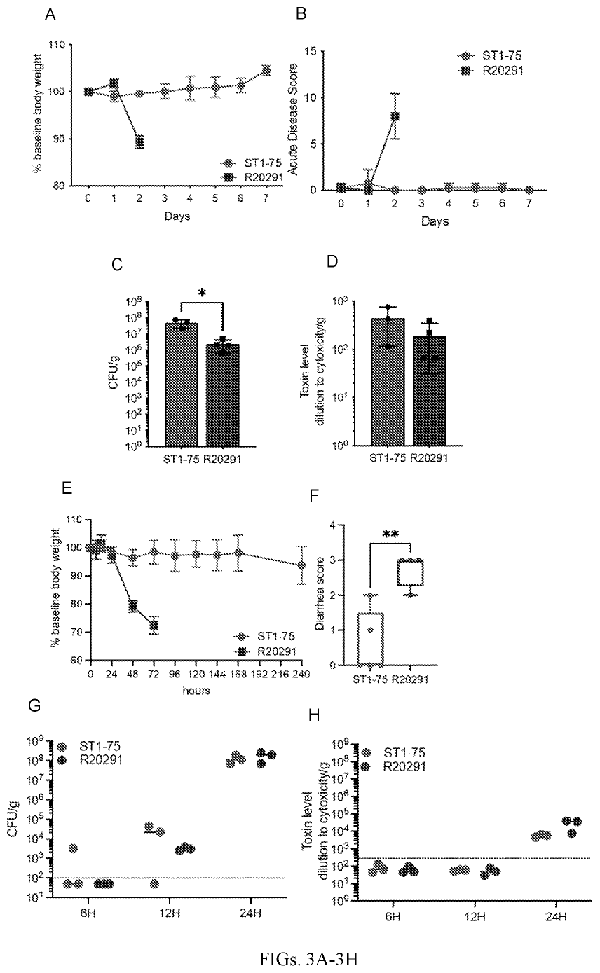

To further investigate this avirulent phenotype, the inventors inoculated ST1-75 into MyD88^{-/-} mice, which lack the adaptor protein for Toll-like receptor signaling.²⁴ MyD88^{-/-} mice fail to recruit neutrophils to the colonic tissue during early stages of C. difficile infection, and display markedly increased susceptibility to C. difficile induced colitis.²⁵ Here, MyD88^{-/-} mice were treated with antibiotics and infected with either ST1-75 or R20291. Mice infected with R20291 quickly succumbed to infection 2 days after spore inoculation, whereas all MyD88^{-/-} mice infected with ST1-75 survived the experiment with minimal weight loss or disease scores (FIG. 3A-3B). Consistent with the results with wildtype mice, no deficiencies of colonization or toxin production were observed day+1 post infection of MyD88^{-/-} mice (FIG. 3C-3D). These data suggest that the attenuation of the avirulent strain is independent of MyD88-mediated host innate immunity.

Germ-free mice are highly susceptible to C. difficile infection because they microbiome-mediated colonization resistance against C. difficile.^{26,27} To investigate whether the gut microbiome renders ST1-75 avirulent, germ-free mice were infected with ST1-75 or R20291. Similarly, the inventors observed no mortality or weight loss in the mice with ST1-75 infection, whereas mice infected with R20291 quickly lost weight and died (or >20% weight loss) (FIG. 3E). Milder diarrhea was observed in mice with ST1-75 compared to R20291 (FIG. 3F). The inventors observed no differences in colonization or fecal toxins between ST1-75 and R20291 up to 24 hours post infection (FIG. 3G-3H). Similar attenuation was also seen in ST1-35 infected germ-free mice (FIG. 9). In contrast, isolates that demonstrated relatively mild pathogenicity in antibiotic-treated mice, such as ST1-25 and ST1-67 (FIG. 1B), led to severe weight loss and diarrhea in germ-free mice (FIG. 9), reaffirming the protective role of the gut microbiome during C. difficile infection. However, the attenuation of ST1-75 and ST1-35 in mice is independent of the gut microbiome.

C. Novel Prophages Identified in Avirulent Strains do not Impact ST1 C. difficile Virulence.

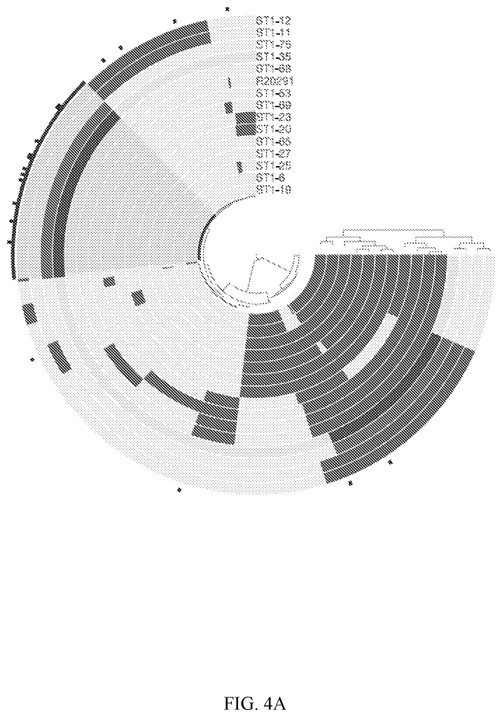

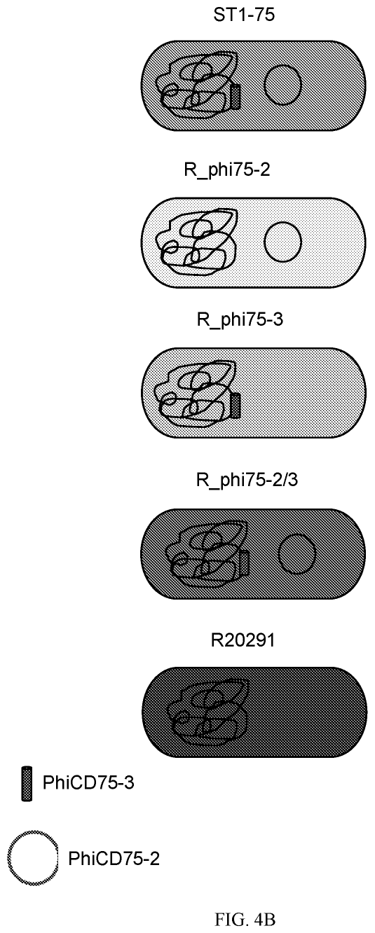

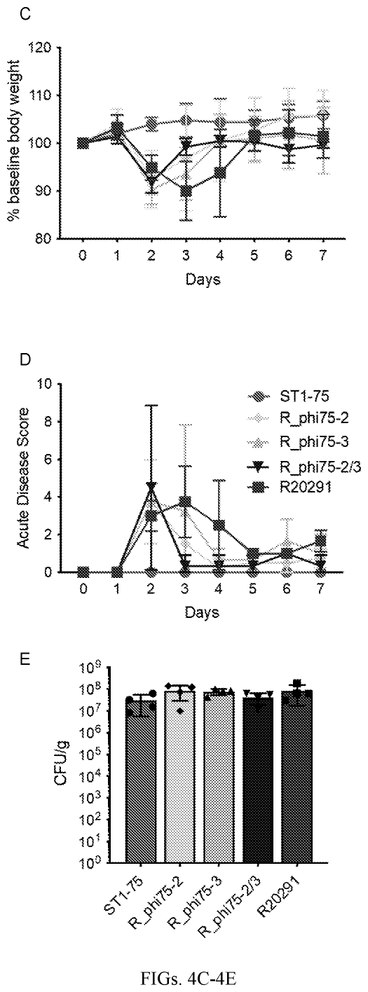

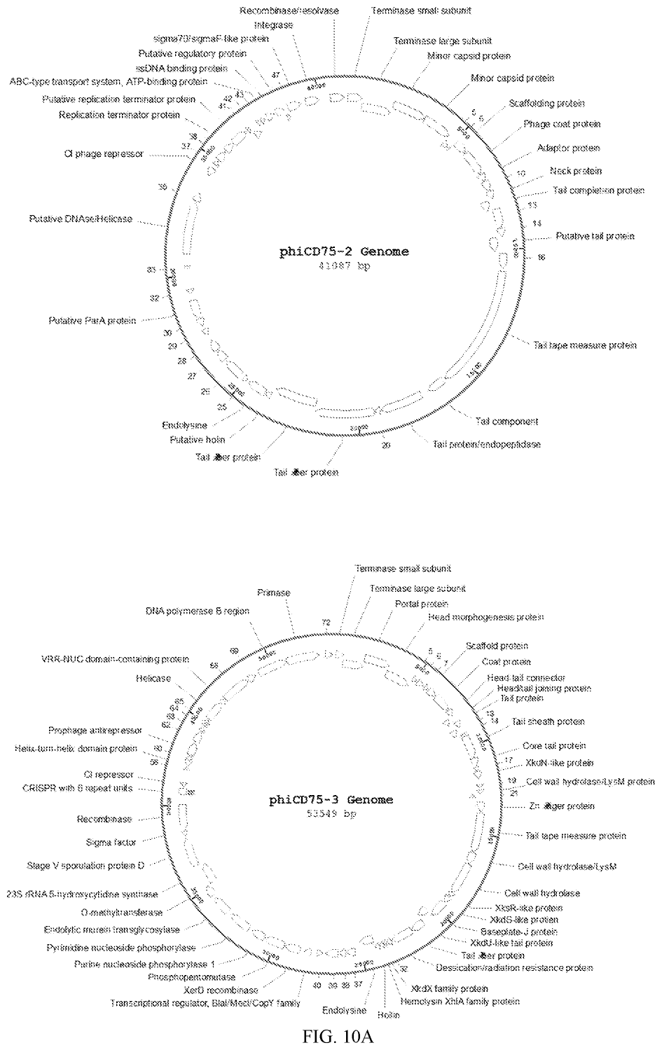

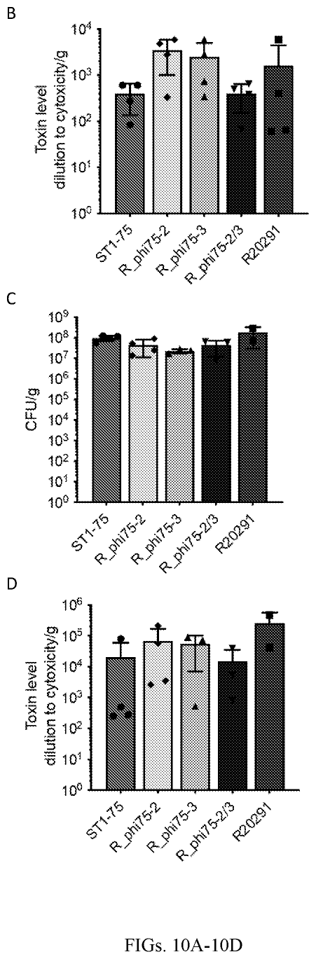

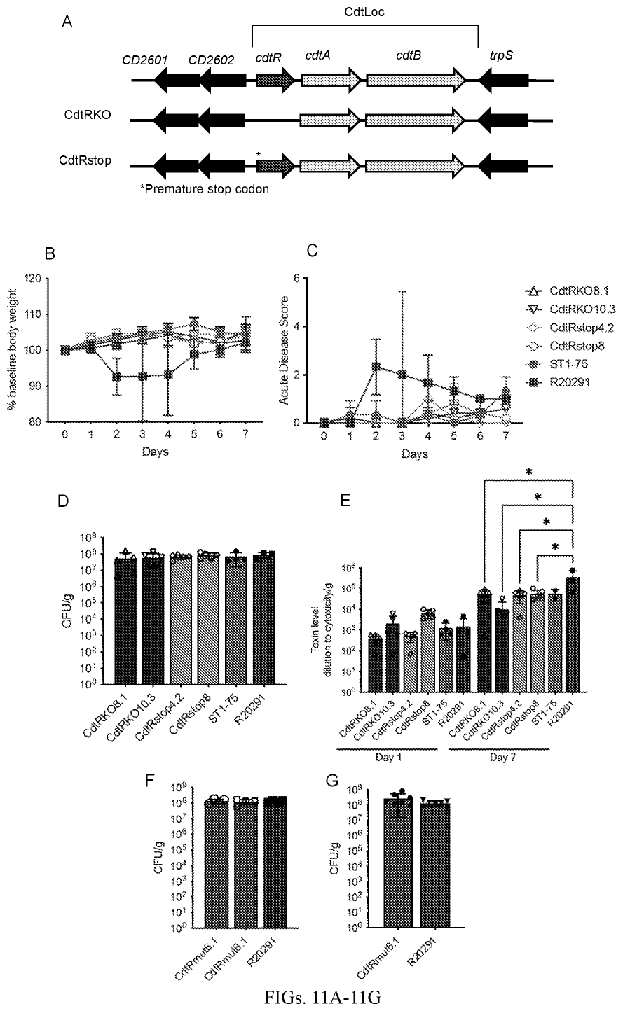

The inventors next sought to determine the genetic factors that may abrogate C. difficile virulence in ST1-75 and ST1-35. Fully circularized genomes of 14 ST1 isolates were successfully obtained using Nanopore and Illumina hybrid assembly, and pangenomic analysis was conducted on these 14 genomes and R20291 using Anvi'o pangenomics workflow.²⁸ A group of gene clusters that is unique to ST1-75 and ST1-35 stood out, which are enriched for phage-related genes (FIG. 4A). The inventors then applied PHASTER, a tool for phage identification in bacterial genomes, to discover two unique prophages in the genomes of ST1-75 and ST1-35. One prophage resides on a 41-kb plasmid in ST1-75 and ST1-35 with 4-5 copies per cell and here is named as phiCD75-2 (FIG. 10A). Blasting phiCD75-2 found high similarities to reported C. difficile phages phiCD38-2 (99.8% identity) and phiCDHS1 (94.7% identity).²⁹⁻³² In addition, a unique 54-kb segment was found as inserted into the chromosomal DNA of ST1-75 and ST1-35 around position 1.29 Mbp and here is named as phiCD75-3 (FIG. 10A). PhiCD75-3 does not show high similarity to any described C. difficile phages to date.

Lysogenic bacteriophages have been identified in many C. difficile genomes, and play an important role in shaping C. difficile evolution. However, their roles in C. difficile biology, especially virulence, are not well-characterized.^{33,34} To investigate the potential role of these two prophages on C. difficile virulence, the inventors induced lytic phage particles of phiCD75-2 and phiCD75-3 from ST1-75 culture and infected R20291 to generate R20291 lysogens harboring these prophages. The inventors were able to generate R20291 derivatives carrying phiCD75-2, phiCD75-3 or both prophages in their genomes (FIG. 4B). Whole-genome sequencing of lysogenic R20291 strains confirmed that phiCD75-3 was inserted in situ as in ST1-75 at 1.29 Mbp. Antibiotic-treated mice infected with R20291 lysogenic strains (FIG. 4B) followed the curve of virulent infection as 10% weight loss and 4-6 disease scores were seen during the peak of symptomatic infection (FIG. 4C-4D). The seemingly faster recovery in the lysogens was not a reproducible finding. Similar levels of colonization and toxin production were also observed (FIGS. 4E and 10B-10D). Disclosed herein are two prophages in avirulent strains ST1-75 and ST1-35, that are not present in R20291 or other ST1 strains from the collection of. However, these two prophages do not appear to impact the virulence of R20291 in antibiotic-treated mice.

D. Mutations in the cdtR Gene Eliminate ST1 C. difficile Virulence in Mice.

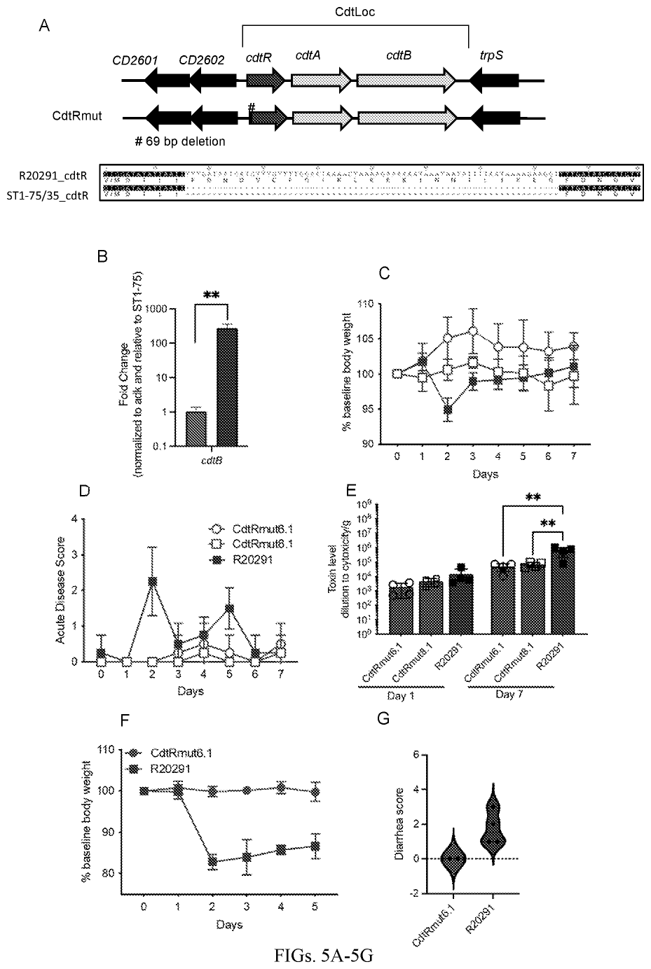

Lysogenic R20291 strains with either or both prophages did not recapitulate the avirulent phenotype of ST1-75 or ST1-35. A closer look at the chromosomal genomes of ST1-75 and ST1-35 led to the discovery of a common mutation in their cdtR gene, which was reported previously as a transcriptional regulator for binary toxin (CDT) genes, cdtA and cdtB.³⁵ A unique 69-bp deletion was found in the cdtR gene of ST1-75 and ST1-35, leading to an in-frame deletion of 23 amino acids (FIG. 5A). To investigate if there is a possible loss of function of CdtR resulted from the deletion, the inventors accessed the transcriptional level of cdtB in mouse cecum following infection of ST1-75 or R20291. More than a 2-log reduction of cdtB transcripts was observed in ST1-75 group (FIG. 5B), suggesting an important role of these 69 base pairs for a fully functional cdtR gene. Next, the inventors applied CRISPR-mediated genome editing approach to generate CdtR mutants using parental R20291 strain to study the contribution of CdtR to C. difficile virulence (FIGS. 11A and 5A). In accordance with a previous report²¹, knocking out cdtR either by deleting the whole gene (CdtRKO8.1 and CdtRKO10.3), or introducing a proximal premature stop codon (CdtRstop4.2 and CdtRstop8) led to a loss of pathogenicity in antibiotic-treated mice (FIG. 11B-11C), confirming a critical role of CdtR for C. difficile virulence. Moreover, deleting the exact same 69-bp region, as in ST1-75/35, in the cdtR of R20291 (CdtRmut6.1 and CdtRmut8.1) again eliminated the virulence of C. difficile (FIG. 5C-5D). Thus, loss of the 69-bp in cdtR explains the avirulence phenotype of ST1-75/35. On the other hand, colonization of these CdtR mutants, assessed by CFU at day+1 post infection, was comparable to that of R20291 (FIGS. 11D and 11F), suggesting that CdtR is not required for colonization. Interestingly, while the fecal levels of TcdA and TcdB of CdtR mutants were comparable to R20291 in the early phase (day+1 post-infection), a significantly reduced level at a later time point (7-days post infection) was observed upon infection of CdtR mutants (FIGS. 11E and 5E), supporting a role of CdtR in regulating PaLoc toxins production. Further, infecting germ-free mice with CdtRmut6.1 results in no weight loss or diarrhea, perfectly recapitulating ST1-75 and ST1-35 phenotypes in germ-free mice (FIGS. 5F-5G and FIG. 11G). Collectively, CRISPR-edited CdtR mutant strains mimic phenotypes of ST1-75 and ST1-35 in mice, demonstrating that cdtR gene is necessary for in vivo virulence. Furthermore, the 69-bp region in cdtR, which is deleted in ST1-75/35, is necessary for proper CdtR function through mechanisms yet to be determined.

E. Mutations in cdtR Reduce PaLoc Toxin Transcription In Vivo

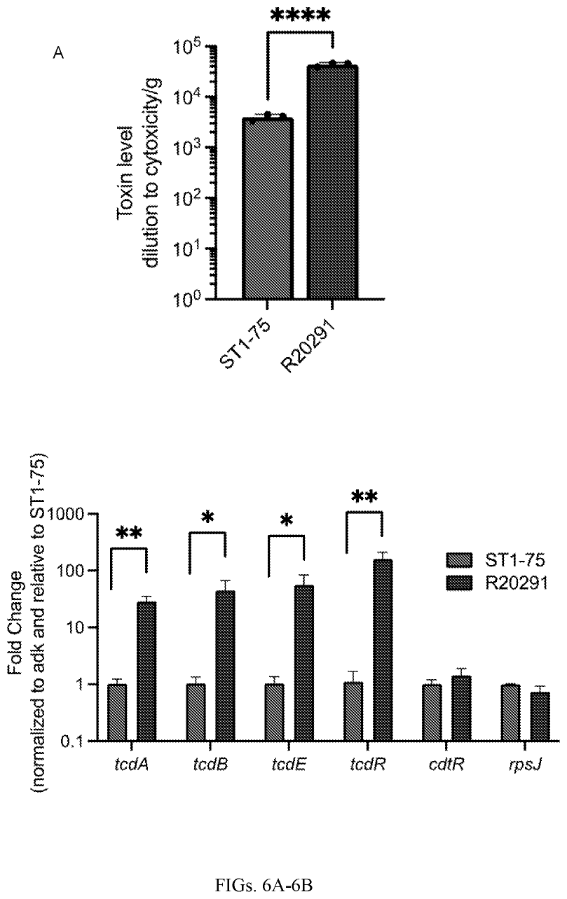

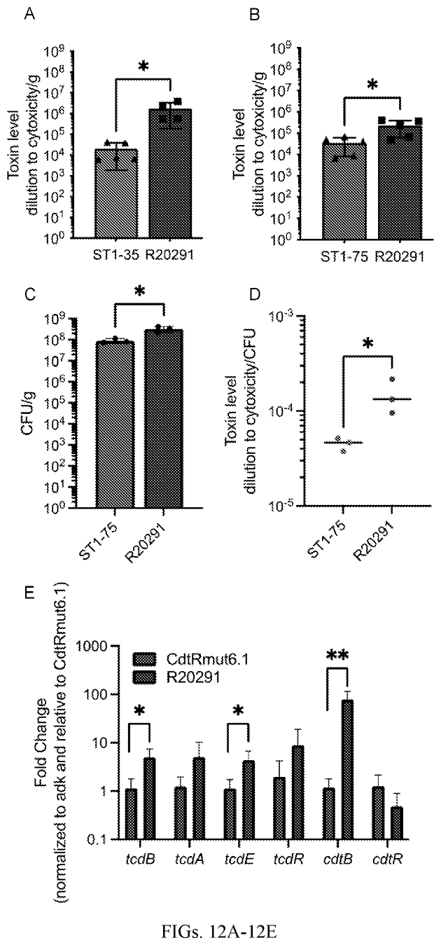

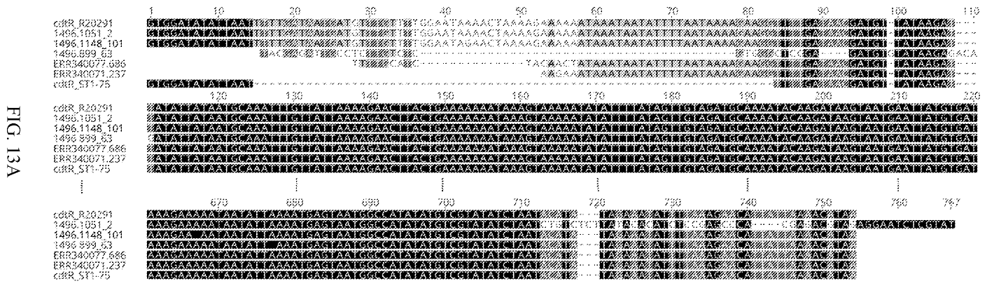

CdtR mutants produce significantly reduced fecal toxins at a later time point (7-days post infection) (FIGS. 5E and 11E), a phenotype that was confirmed in ST1-75 and ST1-35 (FIGS. 12A-12B). To examine whether the 69-bp deletion in cdtR impacts PaLoc toxin production, the inventors harvested cecal contents from germ-free mice infected with ST1-75 or R20291. In contrast to fecal toxin levels, the inventors observed a significantly reduced cecal toxin level in mice infected with ST1-75 compared to R20291 (FIG. 6A). This was not due to a slightly lower CFU of cecal ST1-75 in germ-free mice (FIGS. 12C-12D). The inventors further validated the reduced toxin production in cecal content by RT-qPCR and the inventors observed a 50-fold reduction of the tcdA and tcdB transcripts in the cecum of mice infected with ST1-75 (FIG. 6B). Additionally, transcriptions of other PaLoc genes including tcdE and tcdR were also reduced in the cecum of mice infected with ST1-75 (FIG. 6B). TcdE is a putative holin that mediates toxin secretion.³⁶⁻³⁸ Reduced tcdE likely further impacts the amount of toxins that may reach to the intestinal epithelium. TcdR is a positive regulator of the PaLoc39.40 and is likely the common target of CdtR, which results in the observed downregulation of many PaLoc genes. Interestingly, cdtR transcripts were comparable between ST1-75 and R20291, suggesting that the 69-bp deletion does not impact the transcripts stability but leads to a nonfunctioning product. These results were further confirmed with CdtRmut6.1 strain, though to a lesser extent (FIG. 12E). Collectively, the inventors demonstrated that a natural mutation found in cdtR of two ST1 clinical isolates results in reduced binary toxin production, reduced PaLoc toxin production (and likely secretion) in cecum of infected mice, and attenuated C. difficile virulence, independent of host innate immunity, colonization burden, microbiome constitution, or any noticeable impact of incidentally discovered prophages within these strains. This difference of toxin production in cecum at 24-hour post infection is however, not reflected in feces in parallel, but could be reflected at a later time point, likely due to cumulative differences over time.

F. cdtR is Versatile and More Prevalent than cdtA and cdtB

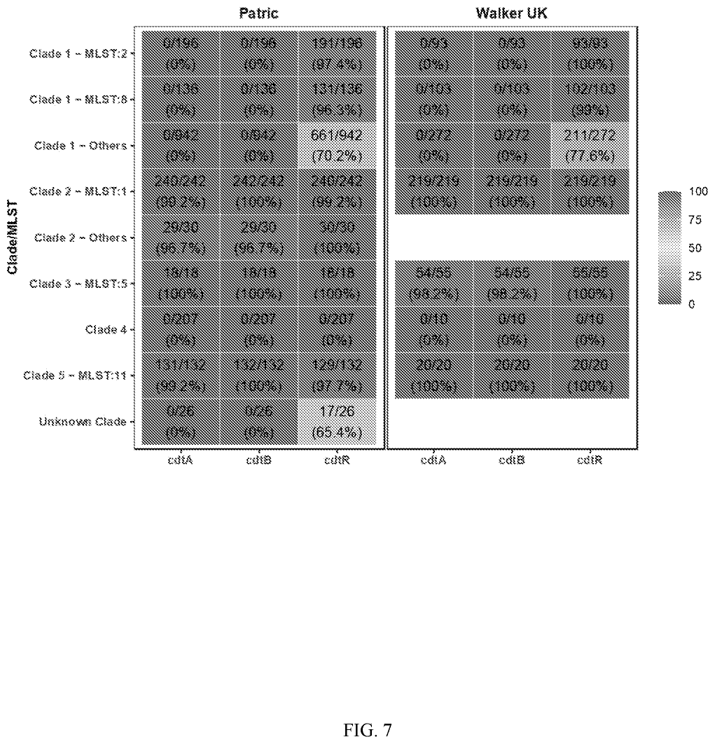

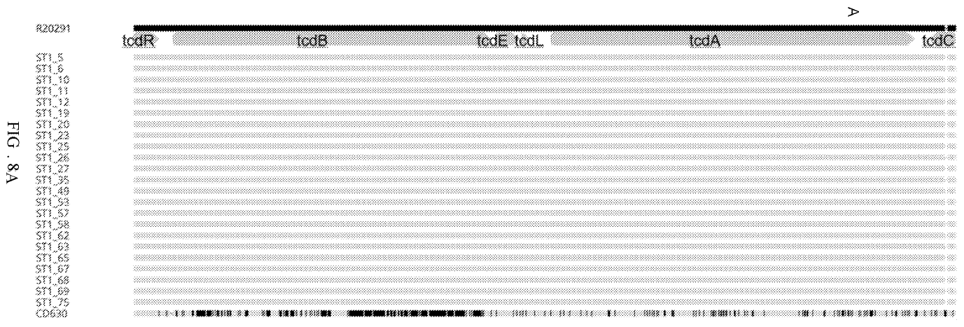

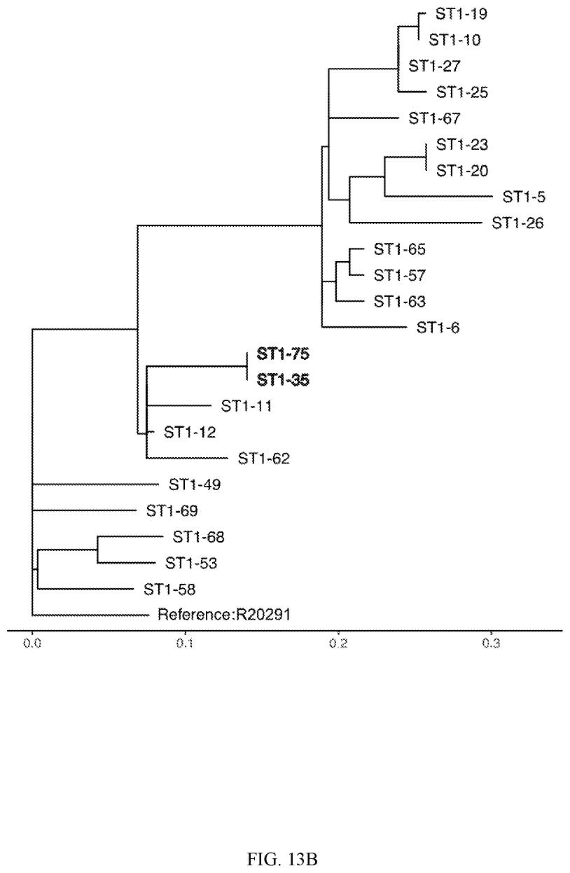

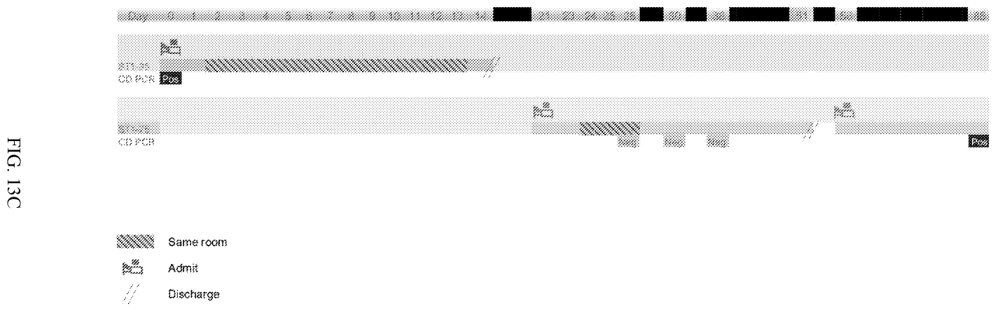

The data support a regulatory role of CdtR outside CdtLoc, so the inventors hypothesize that CdtR may evolve to impact virulence beyond regulating CDT binary toxins. To test this possibility, the inventors surveyed the presence of cdtR, cdtA and cdtB in two major C. difficile clinical collections.^{23,41} As expected, the majority of clade 2 strains, including the epidemic ST1/RT027 strains, contains the CdtLoc with the presence of all three genes. Other subgroups of C. difficile strains, including MLST5 and MLST11 were also reported to encode CDT (FIG. 7A).^{42,43} Unexpectedly, many strain types of C. difficile that were reported as CDT-negative also encode cdtR, such as MLST2, MLST8 from clade 1 (FIG. 7A). The higher prevalence of cdtR over cdtA and cdtB supports the possibility that CdtR functions beyond regulating CDT. Additional work is needed to evaluate the functions of CdtR in these CDT-negative strains. On the contrary, the presence of cdtR in CDT-positive strains may lose its function, such as in MLST11 strains, where a premature stop codon was found and results in a cdtR pseudogene⁴², as well as here in the case of ST1-75/35. To evaluate the prevalence of cdtR mutations that may lead to a loss of function, the inventors aligned all cdtR genes in MLST1 strains from the two described collections and found a few strains having similar truncations at the proximal end and may have lost CdtR function, yet the inventors did not find the exact same deletion as in ST1-75/35 within almost 500 strains (FIG. 13A).

The high similarity between ST1-75 and ST-35 genetically and phenotypically led the inventors reason whether they are clonal. The inventors performed a core-genome SNP analysis across all ST1 isolates from the collection with R20291 as the reference. ST1-75 and ST1-35 shared all SNPs when compared to the genome of R20291 (FIG. 13B). Additional clinical evidence supports ST1-35 and ST1-75 being clonal strains isolated from two patients that shared the same hospital room a few days apart (FIG. 13C). The survey on CdtLoc genes suggest that this locus is very versatile during evolution and cdtR is more prevalent than cdtA and cdtB, which may regulate virulence beyond CDT.

### G. Avirulent C.Difficile Strain Provides Protection Against Virulent Infection in Mice

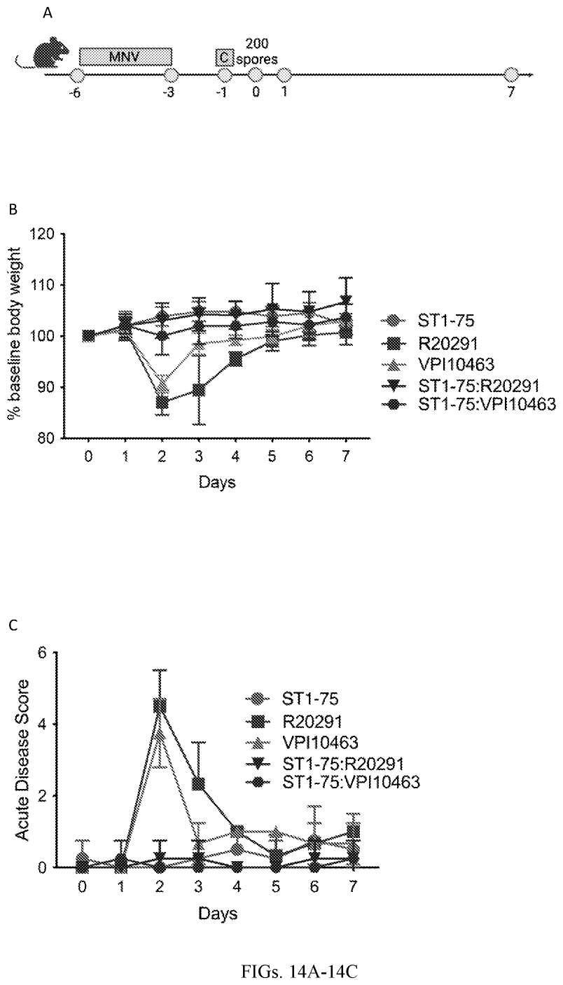

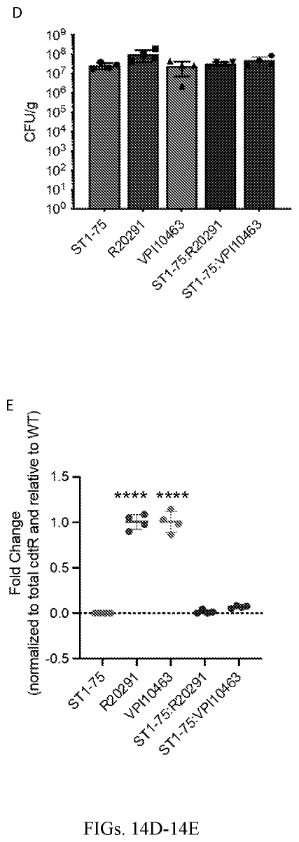

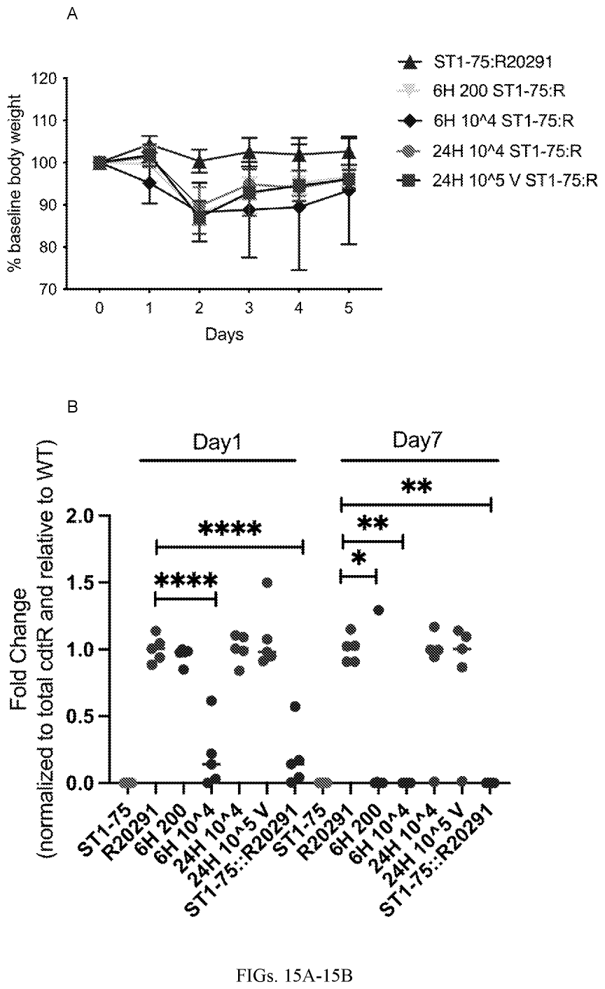

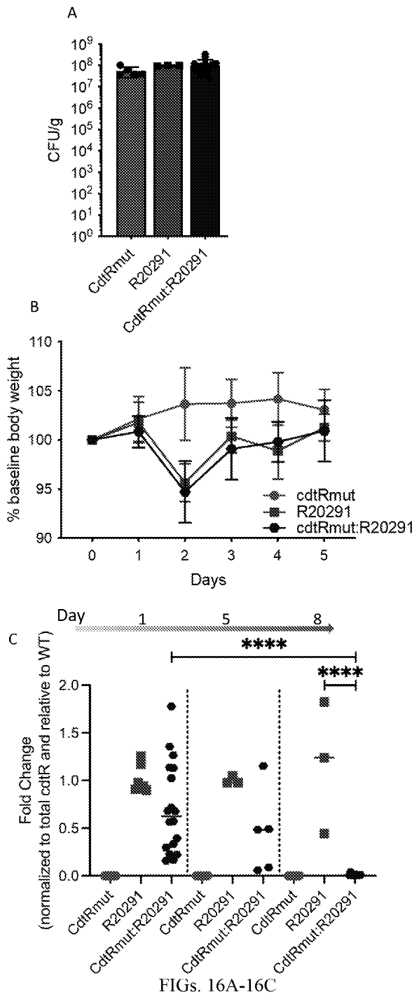

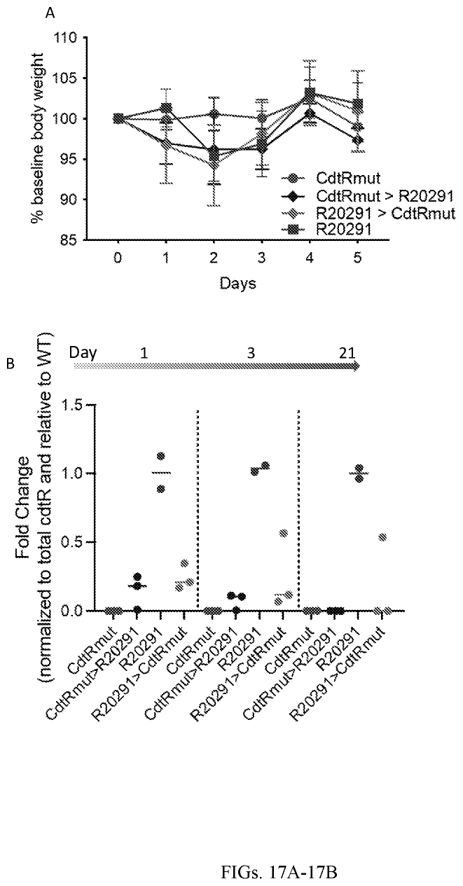

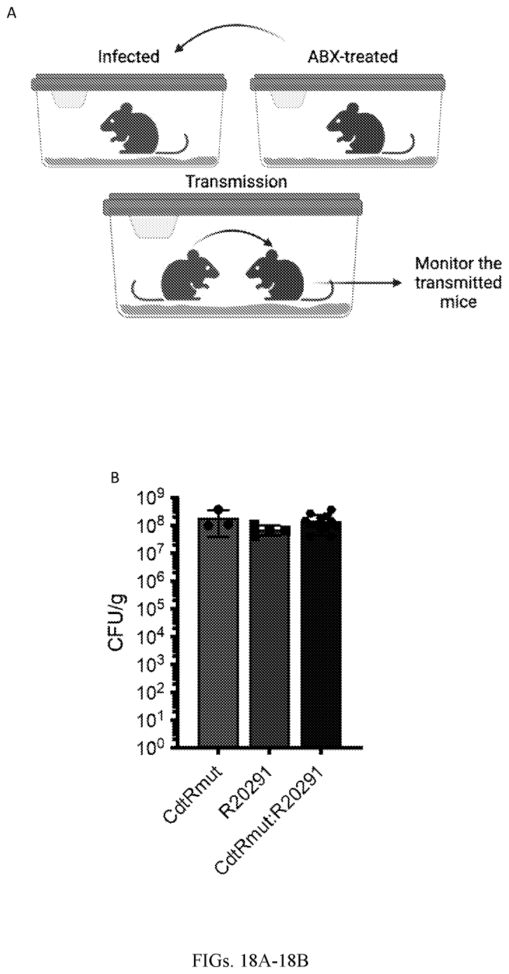

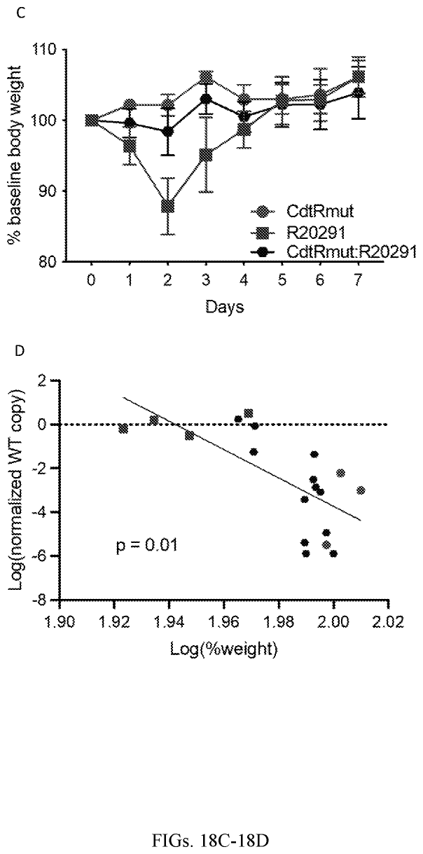

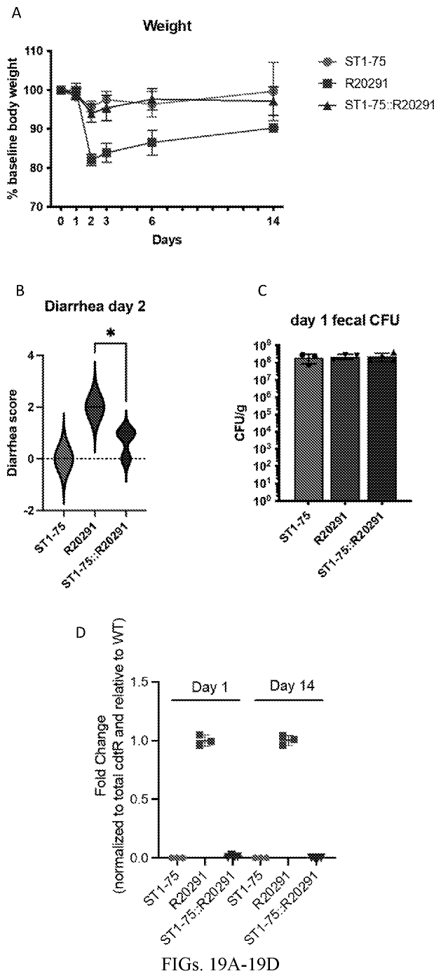

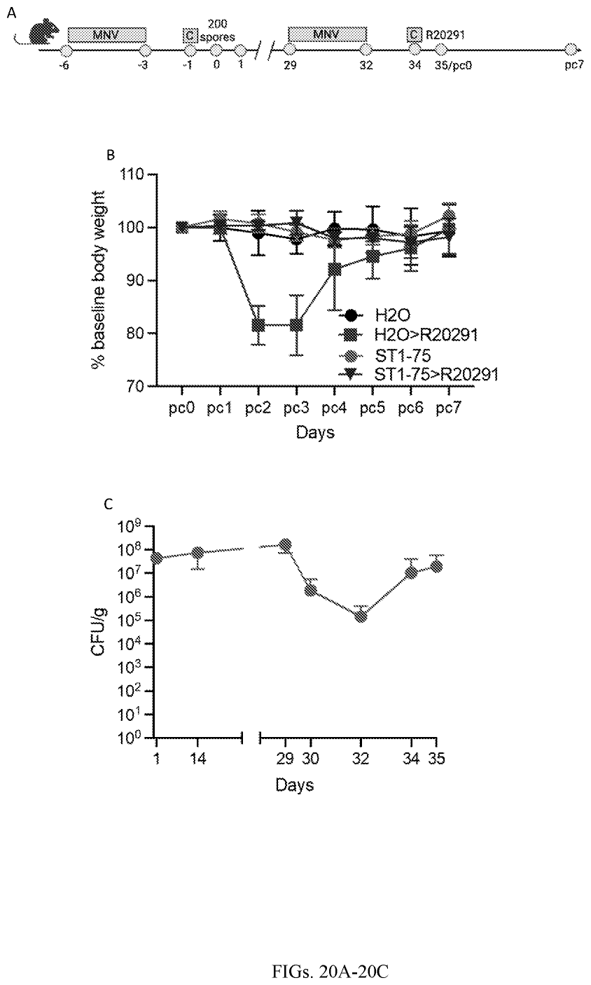

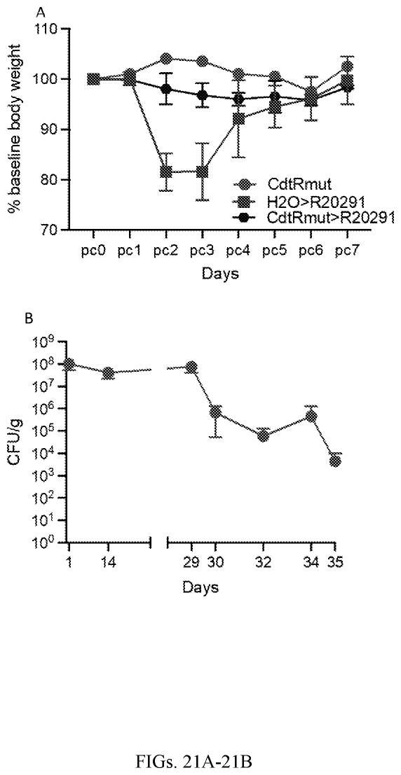

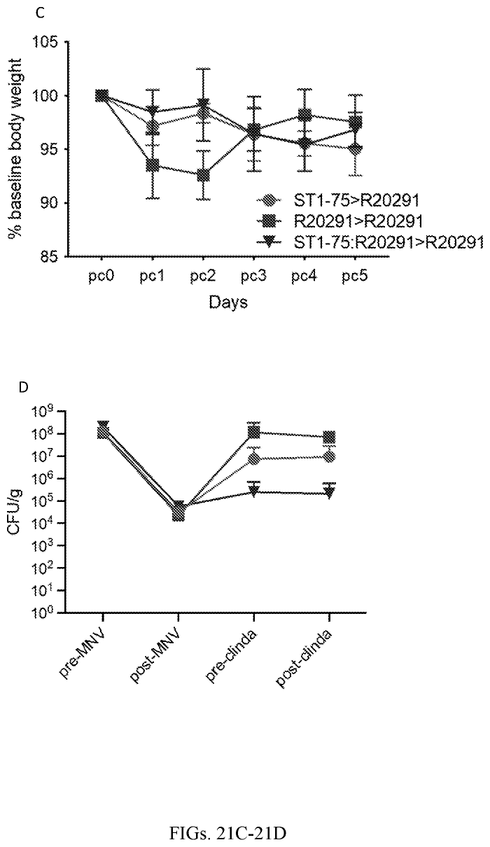

Wildtype C57BL/6 mice (n=4 per group) were treated with metronidazole, vancomycin, and neomycin (MNV, 0.25 g/L for each) in drinking water for 3 days and followed by one intraperitoneal injection of clindamycin (200 μg/mouse) 2 days after antibiotic recess. Then, mice were inoculated with 200 C. difficile spores via oral gavage. In the case of co-infection, mice were inoculated with 100 C. difficile spores for each indicated strains via oral gavage. Daily body weight and acute disease scores were monitored for 7 days post infection (FIGS. 14A-14D). Additionally, two mutant R20291 strains were generated (CdtRmut6.1 (SEQ ID NO:3) and CdtRmut8.1 (SEQ ID NO:4)) with the 69-bp region deleted in the cdtR gene.

H. Discussion on Results and Expectations from Certain Aspects

Mouse models are valuable tools to study how C. difficile strain variations may result in variable disease severities, thanks to the advantages of their identical genetic, immune background and controlled microbiome compositions. Here, the inventors focused on a group of clinical C. difficile isolates belonging to the RT027/MLST1, with high genomic similarity, that all encode PaLoc and CdtLoc. The inventors found that these similar C. difficile isolates caused variable disease severities in mice and that a very specific mutation in the cdtR gene rendered two clinical isolates, ST1-75 and ST1-35, avirulent. Avirulence was solely dependent on the cdtR mutation, as the inventors obtained similar observations using MyD88^{-/-} mice, and germ-free mice, which was also further validated with CRISPR-edited cdtR mutants. Lower transcripts of binary toxin gene cdtB, toxins A tcdA, toxin B tcdB, together with other PaLoc genes including regulator tcdR and putative holin tcdE, were observed in mouse cecum infected with the CdtR mutants. The data support a critical role of CdtR in regulating C. difficile toxin production and secretion, which is essential to ST1 virulence. However, all the other ST1 isolates in this study encoded an intact CdtLoc with wildtype cdtR, whose variations in virulence are likely attributable to alternative mechanisms.

The presence of a binary toxin locus has been associated with epidemic strains and hypervirulence of C. difficile.^{44,45} CDT belongs to the family of ADP-ribosylating toxins that consist of two components: CDTa (cdtA), the enzymatic active ADP-ribosyltransferase which modifies cellular actin, and CDTb (cdtB), the binding component facilitates CDTa translocation. However, despite knowing their enzymatic activities, experimental evidence is very limited to support critical roles of CDT in C. difficile virulence.⁴⁶ CDTb was reported to induce TLR2-dependent pathogenic inflammation, which suppresses a protective cosinophilic response and enhances virulence of RT027 strains, however, C. difficile lacking CDTb still causes acute disease in mice.⁴⁷ On the other hand, CdtR, as the transcriptional regulator of cdtA and cdtB^{6,35,48}, has been previously linked to PaLoc toxin production²¹, suggesting a role as a major virulence regulator. Here, the inventors demonstrated a critical role of CdtR as a determinant of C. difficile virulence within ST1 strains. A natural 69-bp deletion in cdtR that was found in two clinical isolates can reverse the virulence of a wildtype strain, by downregulating the expression of PaLoc genes and binary toxin genes. Additionally, higher prevalence of cdtR over cdtA or cdtB was found while surveying CdtLoc on clinical isolates from public databases. This suggests CdtR may have evolved to function beyond regulating cdtA and cdtB. Systematically examining the target genes of CdtR may give insights on its additional functions, which may also help unveil the mechanisms by which CdtR regulates the PaLoc genes.

ST1-75 and ST1-35 are avirulent in susceptible mouse models despite producing toxins, albeit at reduced levels. This is intriguing because it is well appreciated that toxin expression is necessary for C. difficile virulence.^{46,49} However, the data indicate that toxin production is not sufficient for causing CDI. The amount of toxin being produced and released is likely impact the development of disease. The patients from whom the inventors isolated ST1-75 or ST1-35 had an overall mild clinical assessment, whose symptoms may be attributable to causes other than C. difficile infection. Current CDI diagnoses largely depend on the detection of TcdB gene or toxin B positivity in feces and may lead to overdiagnosis of CDI. The studies suggest the importance of quantifying toxins to evaluate CDI cases.⁵⁰⁻⁵² Incorporating adjunctive biomarkers, such as IL-1β. better distinguishes CDI from asymptomatic carriage and non-CDI diarrhea.⁵³ Here. CdtR regulates both toxin production and secretion, and is essential for C. difficile virulence in mice, suggesting it may serve as an adjunctive biomarker for CDI diagnosis.

Apart from characterizing CdtR, the inventors also identified two prophages in ST1-75 and ST1-35. Prophages have been identified in many C. difficile genomes, and play important roles in shaping C. difficile evolution.³³ While prophages are highly prevalent in C. difficile, little is known about how prophages impact C. difficile biology. A couple of pioneering studies have shown that prophages can affect C. difficile gene expression, impacting toxin production.^{29,54,55} In this study the inventors identified two prophages in ST1-75/35 and named them phiCD75-2 and phiCD75-3. By making R20291 lysogenic strains harboring either or both prophages, the inventors observed minimal impacts on C. difficile virulence by neither of the prophages in antibiotic-treated mice. PhiCD38-2 was shown to increase PaLoc gene expression and toxin production in some RT027 isolates, but not in all of them, suggesting that the genetic background influences the impact of a newly acquired prophage.²⁹ This may explain why phiCD75-2 (a phiCD38-2 derivative) did not increase toxin production ST1-75. Certain phages also impact phase variation of the cell surface protein, biofilm formation, and carry genes involved in quorum sensing, inferring their roles in bacterial fitness.^{30,56,57} One could investigate how phiCD75-2 and phiCD75-3 may impact C. difficile fitness.

In summary, the inventors demonstrate that ST1 C. difficile clinical isolates with identical PaLoc display variable virulence in vivo. Among them, two clonal clinical isolates, ST1-75 and ST1-35, were avirulent in mice, due to a 69-bp deletion mutation in their cdtR genes. These data suggest that specific cdtR genetic variants within the same strain type may predict disease occurrence and severity. Routine detection of these variants may enhance the specificity of NAATs for CDI diagnosis. The data also corroborate recent clinical observations that toxin detection is unreliable as the sole criterion to distinguish between C. difficile infection and colonization.

### I. Material and Methods for Certain Aspects Herein

1. C. difficile Clinical Isolate Collection and Classification

Toxigenic C. difficile-positive stool specimens were collected at Memorial Sloan Kettering Cancer Center between 2013-2017. C. difficile isolates were recovered by plating onto brain heart infusion (BHI) agar plates supplemented with yeast extract, L-cysteine (BHIS), and the antibiotics D-cycloserine and cefoxitin (BHI and yeast extract were from BD Biosciences, and the other components were from Sigma-Aldrich) in an anaerobic chamber (Coylabs). Individual colonies that were able to grow in the presence of these antibiotics and that had the characteristic phenotype of C. difficile were selected, isolated, and subjected to whole-genome sequencing and MLST classification.⁵⁸

### Mouse Husbandry

Wild-type C57BL/6 mice, aged 6 to 8 weeks, were purchased from the Jackson Laboratories. MyD88^{-/-} mice were maintained in augmentin (0.48 g/L and 0.07 mg/L of amoxicillin and clavulanate respectively) in the drinking water in specific-pathogen-free (SPF) facility at the University of Chicago. Germ-free C57Bl/6J mice were bred and maintained in plastic gnotobiotic isolators within the University of Chicago Gnotobiotic Core Facility and fed ad libitum autoclaved standard chow diet (LabDiets 5K67) before transferring to BSL2 room for infection. Mice housed in the BSL2 animal room are fed irradiated feed and provided with acidified water. All mouse experiments were performed in compliance with University of Chicago's institutional guidelines and were approved by its Institutional Animal Care and Use Committee.

2. C. difficile Spore Preparation and Numeration

C. difficile sporulation and preparation was processed as described previously⁵⁹ with minor modifications. Briefly, single colonies of C. difficile isolates were inoculated in deoxygenated BHIS broth and incubated anaerobically for 40-50 days. C. difficile cells were harvested by centrifugation and five washes with ice-cold water. The cells were then suspended in 20% (w/v) HistoDenz (Sigma, St. Louis, MO) and layered onto a 50% (w/v) HistoDenz solution before centrifugating at 15,000×g for 15 minutes to separate spores from vegetative cells. The purified spores pelleted at the bottom were then collected and washed for four times with ice-cold water to remove traces of HistoDenz, and finally resuspended in sterile water. Prepared spores were heated to 60° C. for 20 min to kill vegetative cells, diluted and plated on both BHIS agar and BHIS agar containing 0.1% (w/v) taurocholic acid (BHIS-TA) for numeration. Spore stocks for mouse infection were verified to have less than 1 vegetative cell per 200 spores (as the infection dose).

### 3. Virulence Assessment of Clinical Isolates in Mice

SPF mice were treated with antibiotic cocktail containing metronidazole, neomycin and vancomycin (MNV) in drinking water (0.25 g/L for each antibiotic) for 3 days, 2 days after removing MNV, the mice were received one dose of clindamycin (200 μg/mouse) via intraperitoneal injection. Mice were then the next day infected with 200 C. difficile spores via oral gavage. Germ-free mice were infected with 200 C. difficile spores via oral gavage without antibiotic treatments.

Following infection, mice were monitored and scored for disease severity by four parameters⁶⁰: weight loss (>95% of initial weight=0, 95%-90% initial weight=1, 90%-80% initial weight=2, <80%=3), surface body temperature (>95% of initial temp=0, 95%-90% initial temp=1, 90%-85% initial temp=2, <85%=3), diarrhea severity (formed pellets=0, loose pellets=1, liquid discharge=2, no pellets/caked to fur=3), morbidity (score of 1 for each symptoms with max score of 3; ruffled fur, hunched back, lethargy, ocular discharge).

### 4. Quantification of Fecal Colony Forming Units

Fecal pellets or cecal content from C. difficile infected mice were harvested and resuspended in deoxygenated phosphate-buffed saline (PBS), diluted and plated on BHI agar supplemented with yeast extract, taurocholic acid, L-cysteine, D-cycloserine and cefoxitin (CC-BHIS-TA) at 37° C. anaerobically for overnight.⁶¹

### 5. Cell-Based Assay to Quantify Fecal and Cecal Toxin

The presence of C. difficile toxins was determined using a cell-based cytotoxicity assay as previously described with minor modifications.⁶¹ Briefly, Chinese hamster ovary cells (CHO/dhFr-, ATCC #CRL-9096) were incubated in a 96-well plate overnight at 37° C. Ten-fold dilutions of supernatant from resuspended fecal or cecal content were added to CHO/dhFr-cells, incubated overnight at 37° C. Cell rounding and death was scored the next day. The presence of C. difficile toxins was confirmed by neutralization by antitoxin antisera (Techlab, Blacksburg, VA). The data are expressed as the log 10 reciprocal value of the last dilution where cell rounding was observed.

### 6. DNA Extraction, RNA Extraction and Reverse Transcription

Fecal DNA was extracted using DNeasy PowerSoil Pro Kit (Qiagen), and RNA was isolated from cecal contents using RNeasy PowerMicrobiome Kit (Qiagen) according to the manufacturer's instructions, respectively. Complementary DNA was generated using the QuantiTect reverse transcriptase kit (Qiagen) according to the manufacturer's instructions.

7. Quantitative Polymerase Chain Reaction (qPCR)

Quantitative PCR was performed on genomic DNA or complementary DNA using primers (listed in Table 1) with PowerTrack SYBR Green Master Mix (Thermo Fisher). Reactions were run on a QuantStudio 6 pro (Thermo Fisher). Relative abundance was normalized by ΔΔCt.

TABLE 1
Oligonucleotides used in aspects herein

| | | SEQ ID | | |
|---|---|---|---|---|
| Name | Sequence 5′-3′ | NO | Reference | Purpose |
| tcdA_qFor | GTATGGATAGGTGGAGAAGT | 10 | Babakhani 2012 J | Forward primer |
| | CA | | Antimicrobial | for tcdA |
| | | | Chemotherapy | transcription |
| | | | | analysis |
| tcdA_qRev | CTCTTCCTCTAGTAGCTGTA | 11 | Babakhani 2012 J | Reverse primer |
| | ATGC | | Antimicrobial | for tcdA |
| | | | Chemotherapy | transcription |
| | | | | analysis |
| tcdB_qFor | AGCAGTTGAATATAGTGGTT | 12 | Wroblewski 2009 J | Forward primer |
| | TAGTTAGAGTTG | | Clin Microbiol. | for tcdB |
| | | | | transcription |
| | | | | analysis |
| tcdB_qRev | CATGCTTTTTTAGTTTCTGGA | 13 | Wroblewski 2009 J | Reverse primer |
| | TTGAA | | Clin Microbiol. | for tcdB |
| | | | | transcription |
| | | | | analysis |
| tcdE_qFor | ATAAACCTAGGAGGCGTTAT | 14 | Edwards 2020 J | Forward primer |
| | GAATATGA | | Bacteriology | for tcdE |
| | | | | transcription |
| | | | | analysis |
| tcdE_qRev | TTATTGCACTTAAACATCCT | 15 | Edwards 2020 J | Reverse primer |
| | AATAATGTATCAAA | | Bacteriology | for tcdE |
| | | | | transcription |
| | | | | analysis |
| tcdR_qFor | AGCAAGAAATAACTCAGTA | 16 | Edwards 2020 J | Forward primer |
| | GATGATT | | Bacteriology | for tcdR |
| | | | | transcription |
| | | | | analysis |
| tcdR_qRev | TTATTAAATCTGTTTCTCCCT | 17 | Edwards 2020 J | Reverse primer |
| | CTTCA | | Bacteriology | for tcdR |
| | | | | transcription |
| | | | | analysis |
| cdtB_qFor | GCAGTTAAGTGGGAAGATA | 18 | Angione 2014 J Mol | Forward primer |
| | G | | Diagn | for cdtB |
| | | | | transcription |
| | | | | analysis |
| cdtB_qRev | TCCATACCTACTCCAACAAT | 19 | Angione 2014 J Mol | Reverse primer |
| | | | Diagn | for cdtB |
| | | | | transcription |
| | | | | analysis |
| cdtR-2_qFor | TTGAAACAAGCGCTATTCCA | 20 | This study | Forward primer |
| | CA | | | for cdtR |
| | | | | transcription |
| | | | | analysis |
| cdtR-2_qRev | TGTACACGAATAAAGCATGC | 21 | This study | Reverse primer |
| | | | | for cdtR |
| | ATC | | | transcription |
| | | | | analysis |
| rpsJ_qFor | GATCACAAGTTTCAGGACCT | 22 | Metcalf 2010 | Forward primer |
| | G | | Anaerobe | for rspJ |
| | | | | transcription |
| | | | | analysis |
| rpsJ_qRev | GTCTTAGGTGTTGGATTAGC | 23 | Metcalf 2010 | Reverse primer |
| | | | Anaerobe | for rspJ |
| | | | | transcription |
| | | | | analysis |
| adk_qFor | GTGTATGTGATGTATGCCAA | 24 | Metcalf 2010 | Forward primer |
| | G | | Anaerobe | for adk |
| | | | | transcription |
| | | | | analysis |
| adk_qRev | CCTAAGGCTGCGACAATATC | 25 | Metcalf 2010 | Reverse primer |
| | | | Anaerobe | for adk |
| | | | | transcription |
| | | | | analysis |
| R_cdtR_up_ | GCCTAAACACACATTATCAT | 26 | This study | Forward primer |
| For | AAACAGCTATGACCGCGGCC | | | amplifying |
| | CTCTCTG | | | upstream region |
| | | | | of cdtR gene for |
| | | | | cdtRKO, |
| | | | | cdtRstop and |
| | | | | cdtRmut |
| R_cdtRKO_ | AACTTTCAGTTTAGCGGTCT | 27 | This study | Reverse primer |
| up_Rev | GGGCGCCTAAATACCCTCCT | | | amplifying |
| | ATAAAAAATTCAAAAG | | | upstream region |
| | | | | of cdtR gene for |
| | | | | cdtRKO |
| R_cdtRKO_ | GGCGCCCAGACCGCTAAACT | 28 | This study | Forward primer |
| down_For | GAAAGTTTAAATAGAAAAA | | | amplifying |
| | AGAGATGTCTCAAGATAAG | | | downstream |
| | | | | region of cdtR |
| | | | | gene for |
| | | | | cdtRKO |
| R_cdtRKO_ | TTATTTTTATGCTAGCTCGAG | 29 | This study | Reverse primer |
| down_Rev | TAAGTCTTGTGCATAAATGT | | | amplifying |
| | TATTAGG | | | downstream |
| | | | | region of cdtR |
| | | | | gene for |
| | | | | cdtRKO |
| R_cdtRstop_ | AACTTTCAGTTTAGCGGTCT | 30 | This study | Reverse primer |
| up_Rev | GGGCGCCTTATCAAAAATTA | | | amplifying |
| | ATATATCCACTAAATACCC | | | upstream region |
| | | | | of cdtR gene for |
| | | | | cdtRstop |
| R_cdtRstop_ | GGCGCCCAGACCGCTAAACT | 31 | This study | Forward primer |
| down_For | GAAAGTTTTTTGATAACGAT | | | amplifying |
| | GTTATAAGATTATATTATAA | | | downstream |
| | | | | region of cdtR |
| | | | | gene for |
| | | | | cdtRstop |
| R_cdtRstop/ | TTATTTTTATGCTAGCTCGAG | 32 | This study | Reverse primer |
| mut_down_ | ATCTGATAAAGACCTTAAAC | | | amplifying |
| Rev | TTTTATAG | | | downstream |
| | | | | region of cdtR |
| | | | | gene for |
| | | | | cdtRstop and |
| | | | | cdtRmut |
| R_cdtRmut_ | TTATAATATAATCTTATAAC | 33 | This study | Reverse primer |
| up_Rev | ATCGTTATCAAAAATTAATA | | | amplifying |
| | TATCCACTAAATACCC | | | upstream region |
| | | | | of cdtR gene for |
| | | | | cdtRmut |
| R_cdtRmut_ | GGGTATTTAGTGGATATATT | 34 | This study | Forward primer |
| down_For | AATTTTTGATAACGATGTTA | | | amplifying |
| | TAAGATTATATTATAA | | | downstream |
| | | | | region of cdtR |
| | | | | gene for |
| | | | | cdtRmut |
| R_cdtR_sgR | aattaaactgtaaatggccaAAT | 35 | This study | Forward primer |
| NA1c_For | AATATTTTAATAAAAGAGTTTTA | | | gRNA Cloning |
| | GAGCTAGAAATAGC | | | into pCE677 |
| | | | | digested with |
| | | | | MscI and MluI |
| | | | | Universal |
| CDEP3876 | aaccatctaaaaatagttgcaga | 36 | Kaus 2020 J | Reverse primer |
| | gcttACGCGTC | | Bacteriol. | for gRNA |
| | | | | Cloning into |
| | | | | pCE677 |
| | | | | digested with |
| | | | | MscI and MluI |
| LCF0312 | AGCGGTATCGGCTTGGTTGT | 37 | This study | Verify phage |
| | AGAT | | | identity for |
| | | | | phiCD75-2 |
| LCF0313 | TGCTAGTTTCCTGTCAAGGT | 38 | This study | Verify phage |
| | CGCT | | | identity for |
| | | | | phiCD75-2 |
| LCF1242 | CGACCCACCTAAAGGTATTC | 39 | This study | Verify phage |
| | A | | | identity for |
| | | | | phiCD75-3 |
| LCF1243 | GTTCTTTAGTCCAGTTCCCAT | 40 | This study | Verify phage |
| | TTC | | | identity for |
| | | | | phiCD75-3 |

8. Generation of C. difficile cdtR Mutants Using CRISPR

CRISPR editing on C. difficile strains R20291 was performed as described in.⁶² The primers were listed in Table S1⁶³⁻⁶⁷. Briefly, donor regions for homology were generated by separately amplifying regions ˜500 bp upstream and ˜500 bp downstream of the target of interest. The resulting regions were cloned into pCE677 between NotI and XhoI sites by Gibson Assembly. Geneious Prime (v11) was used to design sgRNAs targeting each deleted target. sgRNA fragments were then amplified by PCR from pCE677, using an upstream primer that introduces the altered guide and inserted at the MscI and MluI sites of the pCE677-derivative with the appropriate homology region. Regions of plasmids constructed using PCR were verified by Sanger sequencing. Plasmids were then passaged through NEBturbo E. coli strain before transformation into Bacillus subtilis strain BS49. The CRISPR-Cas9 deletion plasmids which harbor the oriT (Tn916) origin of transfer, were then introduced into C. difficile strains by conjugation.⁶⁸ C. difficile colonies were then screened for proper mutations in the genomes by PCR and correct clones were further validated by whole-genome sequencing.

### 9. Whole-Genome Sequencing and Assembly

DNA was extracted using the QIAamp PowerFecal Pro DNA kit (Qiagen). Libraries were prepared using 100 ng of genomic DNA using the QIAseq FX DNA library kit (Qiagen). Briefly, DNA was fragmented enzymatically into smaller fragments and desired insert size was achieved by adjusting fragmentation conditions. Fragmented DNA was end repaired and ‘A's’ were added to the 3′ ends to stage inserts for ligation. During ligation step, Illumina compatible Unique Dual Index (UDI) adapters were added to the inserts and prepared library was PCR amplified. Amplified libraries were cleaned up, and QC was performed using Tapestation 4200 (Agilent Technologies). Libraries were sequenced on an Illumina NextSeq 500 or MiSeq platform to generate 2×150 or 2×250 bp reads respectively. Illumina reads were assembled into contigs using SPAdes⁶⁹ and genes were called and annotated using Prokka (v1.14.6).⁷⁰

Samples for Nanopore and Illumina hybrid assemblies were extracted using the NEB Monarch Genomic DNA Purification Kit. DNA was QC′ed using genomic Tapestation 4200. Nanopore libraries were prepared using the Ligation Sequencing Kit (SQK-LSK109), the Native Barcoding Expansions 1-12 (EXP-NBD104) and 13-24 (EXP-NBD114), and the NebNext Companion Module for Oxford Nanopore Technologies (E7180S). The shearing steps and first ethanol wash were eliminated to ensure high concentrations of long fragments. Using R9.4.1 flow cells, libraries were run on a MinION for 72 hours at −180V. The Nanopore and Illumina hybrid assemblies were completed using Unicycler (v0.4.8) 71 either with the untrimmed or trimmed Illumina reads. The assemblies with less number of circularized contigs were used for genome analysis.

### 10. Multiple Sequence Alignment for Pathogenicity Loci

Illumina whole-genome of twenty-five C. difficile strains including two reference genome: R20291 (accession: FN545816.1) and CD630 (accession: NC_009089.1), along with 23 in-house strains ST1-10, ST1-11, ST1-12, ST1-19, ST1-20, ST1-23, ST1-25, ST1-26, ST1-27, ST1-35, ST1-49, ST1-5, ST1-53, ST1-57, ST1-58, ST1-6, ST1-62, ST1-63, ST1-65, ST1-67. ST1-68, ST1-69, and ST1-75 were included in the pathogenicity locus (PaLoc) analysis. The PaLoc region of R20291 (NCBI accessions NC_013316, 706,660-725,022 bp) were extracted as the query to BLAST⁷² against a local database of all the above genomes. Hits with at least 85% query coverage and 85% percent identity were extracted and multiple sequence alignment were performed using Gencious Prime 2022.0.1 with default settings to compare their nucleotide differences.

### 11. Binary Toxin Genes Prevalence Analysis

C. difficile isolates (N=827) from BioProject PRJEB4556 were downloaded from NCBI, and assembled into contigs using SPAdes.⁶⁹ A collection of 2143 C. difficile genomes from Patric (date: Feb. 10, 2021)²³ were also downloaded. MLST was determined on those contigs by mlst.⁷³ ST type with less than 3 isolates were removed. Binary toxin cdtA, cdtB and cdtR from R20291 (NCBI accessions NC_013316) were used as query to BLAST⁷² against the assembled contigs, and hits with at least 85% identity and 85% coverage of the query are considered a valid match.

### 12. UMAP (Uniform Manifold Approximation and Projection) Analysis

A subset of isolate contigs of 199 ST1, 50 ST2, 50 ST3, 49 ST6, 50 ST8, 50 ST11, 42 ST14, 50 ST15, 50 ST17, 50 ST37 and 50 ST42, totaling 690 isolates were selected from the above Patric collection. They were all sequenced by short read technology, and they are the top 10 abundant ST groups except ST1 in the Patric collection. Genes were called and annotated from their contigs using Prokka (v1.14.6).⁷⁰ By combining the 23 isolates from this study, the inventors constructed a matrix of 731 isolates by 8025 annotated genes and hypothetical protein clusters. Specifically, hypothetical protein clusters were formed by clustering hypothetical proteins at 50% identity using cd-hit.^{74,75} Any protein sequences that were at least 50% similar fall into an artificially cluster. UMAP analysis was performed on the basis of the presence/absence of the genes/hypothetical protein clusters by setting the n_neighbors parameter to 675.

### 13. Core-Genomes SNPs Analysis on ST1 Isolates

SNP analysis was done on the 23 ST1 isolates from the collection against R20291 using snippy (v4.6.0).⁷⁶ Then the core SNP was extracted, recombination removed using gubbins (v2.4.1)⁷⁷, and a phylogenetic tree was built using fastTree (v2.1.10).^{78,79}

14. MLST1 cdtR SNPs Analysis

cdtR hits without starting position at the beginning of C. difficile contigs were chosen to further examine their nucleotide differences in cdtR gene to R20291 and ST1-75. Five such isolates were found either from Patric collection or BioProject PRJEB4556^{23,41}, and multiple sequences alignment were performed in Geneious Prime 2022.0.1 with default settings.

### 15. FastANI

Genomic similarity between clinical isolates were calculated using FastANI (v 1.32)⁸⁰ and presented as ANI score.

### 16. Pangenomic Analysis of ST1 Isolates Using Anvi'o

Fifteen circularized genomes of ST1 isolates generated by the Nanopore and Illumina hybrid assemblies were used for pangenomic analysis. Default settings were used based on the Anvi'o workflow for microbial pangenomcis with adjustments for minbit as 0.5 and mcl-inflation as 10.^{28,81-83} Annotations were performed with NCBI Clusters of Orthologous Genes (COG).⁸⁴ Accessory genomes were grouped by gene clusters that are not present in all 15 isolate genomes.

### 17. Prophage Identification Using PHASTER

Three complete prophages were identified in the genome sequence of strain ST1-75 using PHASTER.⁸⁵ Based on Blastn analyses, the phiCD75-1 prophage corresponds to phi027, a prophage highly conserved among R027 isolates.⁸⁶ The phiCD75-2 prophage is homologous at 99.82% to the well-described phiCD38-2 phage²⁹, whereas the phiCD75-3 prophage seems to be a new phage with no close homolog in public databases. The detection of ORF and gene annotation on phage genomes were performed with PROKKA 70, using an E-value threshold of 10E-3 for function assignment. The most recent PHASTER database (last update Dec. 22, 2020) was implemented into PROKKA to improve function prediction and overall annotation quality of phage proteins.⁸⁵ The genomes were reorganized so that they started with the terminase gene. Genomic maps were generated using Benchling and finalized with Inkscape v1.2.

### 18. Prophage Induction and Phage Amplification

To confirm the functionality of the phiCD75-2 and phiCD75-3 prophages, induction was performed in TY (2% yeast extract, 3% tryptose, pH 7.4) using two different strategies described previously. The first method was a treatment with 2.5 μg/mL mitomycin C (Novus Biologicals), and the second one was UV irradiation for 10 sec at a wavelength of 365 nm.⁸⁷ The induced cultures were clarified by centrifugation, then filtered on a 0.22 μm filter and the presence of infectious phage particles was confirmed by plating on bacterial lawns of the R20291 epidemic strain using a soft agar overlay method.⁸⁷ Six isolated phage plaques obtained with each induction lysate were picked, diffused in 500 μL phage buffer (50 mM Tris-HCl pH 7.5, 100 mM NaCl, 8 mM MgSO⁴) and the identity of the induced phages was determined by PCR using primers specific for phiCD75-2 and phiCD75-3. Mitomycin C treatment led to induction of the phiCD75-3 prophage whereas UV treatment led to induction of the phiCD75-2 prophage. Each phage was then plaque-purified 3 times to obtain pure phage cultures. Amplification to high titers (>10⁸ pfu/mL) was done in TY broth using strain R20291 as the host and methods described elsewhere.²⁹ Phage genomic DNA was extracted from each lysate and a restriction profile was established using XbaI and HindIII to further confirm the identity of the phages, as done before.²⁹

### 19. Creation of New Lysogens

New lysogens of strain R20291 carrying either phiCD75-2, phiCD75-3 or both phages were created using a method described before.²⁹ Briefly, soft agar overlays containing high titers (>10⁸ pfu/mL) of phage phiCD75-2, phiCD75-3, or both phages in equivalent amounts were prepared and bacterial dilutions of the wildtype R20291 strain were spread on top of the lawn. Phage-resistant colonies that grew after overnight incubation were picked, re-streaked 3 times on TY agar, and the presence of the respective prophage was detected by PCR and by confirming the phage-resistant phenotype upon re-infection with the corresponding phage(s).

### 20. Quantification and Statistical Analysis

Results represent means±SD. Statistical significance was determined by the unpaired t test and one-way ANOVA test. Statistical analyses were performed using Prism GraphPad software v9.3.1 (* p<0.05; ** p<0.01; *** p<0.001; **** p<0.0001

### 21. Data Availability

Whole-genome sequence data were uploaded to National Center for Biotechnology Information (NCBI) Sequence Read Archive (SRA) under BioProject accessions PRJNA885086 and PRJNA595724.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Lessa, F. C., Mu, Y., Bamberg, W. M., Beldavs, Z. G., Dumyati, G. K., Dunn, J. R., Farley, M. M., Holzbauer, S. M., Meck, J. I., Phipps, E. C., et al. (2015). Burden of Clostridium difficile Infection in the United States. New England Journal of Medicine 372, 825-834, 10.1056/NEJMoa1408913.
2. Guh, A. Y., Mu, Y., Winston, L. G., Johnston, H., Olson, D., Farley, M. M., Wilson, L. E., Holzbauer, S. M., Phipps, E. C., Dumyati, G. K., et al. (2020). Trends in U. S. Burden of Clostridioides difficile Infection and Outcomes. New England Journal of Medicine 382, 1320-1330. 10.1056/NEJMoa1910215.
3. Abt, M. C., McKenney, P. T., and Pamer, E. G. (2016). Clostridium difficile colitis: pathogenesis and host defence. Nat. Rev. Microbiol. 14, 609-620. 10.1038/nrmicro.2016.108.
4. Rupnik, M., Wilcox, M. H., and Gerding, D. N. (2009). Clostridium difficile infection: new developments in epidemiology and pathogenesis. Nat Rev Microbiol 7, 526-536. 10.1038/nrmicro2164.
5. Cohen, S. H., Tang, Y. J., and Silva, J. (2000). Analysis of the pathogenicity locus in Clostridium difficile strains. J Infect Dis 181, 659-663. 10.1086/315248.
6. Kordus, S. L., Thomas, A. K., and Lacy, D. B. (2022). Clostridioides difficile toxins: mechanisms of action and antitoxin therapeutics. Nat Rev Microbiol 20, 285-298. 10.1038/s41579-021-00660-2.
7. Knight, D. R., Elliott, B., Chang, B. J., Perkins, T. T., and Riley, T. V. (2015). Diversity and Evolution in the Genome of Clostridium difficile. Clin Microbiol Rev 28, 721-741. 10.1128/CMR.00127-14.
8. Rao, K., Micic, D., Natarajan, M., Winters, S., Kiel, M. J., Walk, S. T., Santhosh, K., Mogle, J. A., Galecki, A. T., LeBar, W., et al. (2015). Clostridium difficile ribotype 027: relationship to age, detectability of toxins A or B in stool with rapid testing, severe infection, and mortality. Clin Infect Dis 61, 233-241. 10.1093/cid/civ254.
9. Sec, I., Mu, Y., Cohen, J., Beldavs, Z. G., Winston, L. G., Dumyati, G., Holzbauer, S., Dunn, J., Farley, M. M., Lyons, C., et al. (2014). NAP1 Strain Type Predicts Outcomes From Clostridium difficile Infection. Clinical Infectious Diseases 58, 1394-1400. 10.1093/cid/ciu125.
10. Giancola, S. E., Williams, R. J., and Gentry, C. A. (2018). Prevalence of the Clostridium difficile BI/NAP1/027 strain across the United States Veterans Health Administration. Clin Microbiol Infect 24, 877-881. 10.1016/j.cmi.2017.11.011.
11. C. difficile Infections | A. R. & Patient Safety Portal https://arpsp.cdc.gov/profile/cip/cdi.
12. Gerding, D. N., Johnson, S., Rupnik, M., and Aktories, K. (2014). Clostridium difficile binary toxin CDT. Gut Microbes 5, 15-27. 10.4161/gmic.26854.
13. Gargis, A. S., Karlsson, M., Paulick, A. L., Anderson, K. F., Adamczyk, M., Vlachos, N., Kent, A. G., McAllister, G., Mckay, S. L., Halpin, A. L., et al. (2022). Reference Susceptibility Testing and Genomic Surveillance of Clostridioides difficile, United States, 2012-17. Clinical Infectious Diseases, ciac817. 10.1093/cid/ciac817.
14. Warny, M., Pepin, J., Fang, A., Killgore, G., Thompson, A., Brazier, J., Frost, E., and McDonald, L. C. (2005). Toxin production by an emerging strain of Clostridium difficile associated with outbreaks of severe disease in North America and Europe. Lancet 366, 1079-1084. 10.1016/S0140-6736 (05) 67420-X.
15. Cloud, J., Noddin, L., Pressman, A., Hu, M., and Kelly, C. (2009). Clostridium difficile strain NAP-1 is not associated with severe disease in a nonepidemic setting. Clin Gastroenterol Hepatol 7, 868-873.c2. 10.1016/j.cgh.2009.05.018.
16. Walk, S. T., Micic, D., Jain, R., Lo, E. S., Trivedi, I., Liu, E. W., Almassalha, L. M., Ewing, S. A., Ring. C., Galecki, A. T., et al. (2012). Clostridium difficile ribotype does not predict severe infection. Clin Infect Dis 55, 1661-1668. 10.1093/cid/cis786.
17. Bauer, K. A., Johnston, J. E. W., Wenzler, E., Goff, D. A., Cook, C. H., Balada-Llasat, J.-M., Pancholi, P., and Mangino, J. E. (2017). Impact of the NAP-1 strain on disease severity, mortality, and recurrence of healthcare-associated Clostridium difficile infection. Anacrobe 48, 1-6. 10.1016/j.anacrobe.2017.06.009.
18. Revolinski, S. L., and Munoz-Price, L. S. (2019). Clostridium difficile in Immunocompromised Hosts: A Review of Epidemiology, Risk Factors, Treatment, and Prevention. Clin Infect Dis 68, 2144-2153. 10.1093/cid/ciy845.
19. Seckatz, A. M., and Young, V. B. (2014). Clostridium difficile and the microbiota. J Clin Invest 124, 4182-4189. 10.1172/JCI72336.
20. Lewis, B. B., Carter, R. A., Ling. L., Leiner, I., Taur, Y., Kamboj, M., Dubberke, E. R., Xavier, J., and Pamer, E. G. (2017). Pathogenicity Locus, Core Genome, and Accessory Gene Contributions to Clostridium difficile Virulence. mBio 8, e00885-17. 10.1128/mBio.00885-17.
21. Lyon, S. A., Hutton, M. L., Rood, J. I., Cheung, J. K., and Lyras, D. (2016). CdtR Regulates TcdA and TcdB Production in Clostridium difficile. PLOS Pathog 12, e1005758. 10.1371/journal.ppat. 1005758.
22. Miles-Jay, A., Young, V. B., Pamer, E. G., Savidge, T. C., Kamboj, M., Garey, K. W., and Snitkin, E. S. (2021). A multisite genomic epidemiology study of Clostridioides difficile infections in the USA supports differential roles of healthcare versus community spread for two common strains. Microb Genom 7, 000590. 10.1099/mgen.0.000590.
23. Wattam, A. R., Davis, J. J., Assaf, R., Boisvert, S., Brettin, T., Bun, C., Conrad, N., Dietrich, E. M., Disz, T., Gabbard, J. L., et al. (2017). Improvements to PATRIC, the all-bacterial Bioinformatics Database and Analysis Resource Center. Nucleic Acids Res. 45, D535-D542. 10.1093/nar/gkw1017.
24. Lebeis, S. L., Bommarius, B., Parkos, C. A., Sherman, M. A., and Kalman, D. (2007). TLR signaling mediated by MyD88 is required for a protective innate immune response by neutrophils to Citrobacter rodentium. J Immunol 179, 566-577. 10.4049/jimmunol.179.1.566.
25. Jarchum, I., Liu, M., Shi, C., Equinda, M., and Pamer, E. G. (2012). Critical role for MyD88-mediated neutrophil recruitment during Clostridium difficile colitis. Infect. Immun. 80, 2989-2996. 10.1128/IAI.00448-12.
26. Buffic, C. G., Bucci, V., Stein, R. R., McKenney, P. T., Ling, L., Gobourne, A., No, D., Liu, H., Kinnebrew, M., Viale, A., et al. (2015). Precision microbiome reconstitution restores bile acid mediated resistance to Clostridium difficile. Nature 517, 205-208. 10.1038/nature13828.
27. Reeves, A. E., Koenigsknecht, M. J., Bergin, I. L., and Young, V. B. (2012). Suppression of Clostridium difficile in the gastrointestinal tracts of germfree mice inoculated with a murine isolate from the family Lachnospiraceae. Infect Immun 80, 3786-3794. 10.1128/IAI.00647-12.
28. Eren, A. M., Kiefl, E., Shaiber, A., Veseli, I., Miller, S. E., Schechter, M. S., Fink, I., Pan, J. N., Yousef, M., Fogarty, E. C., et al. (2021). Community-led, integrated, reproducible multi-omics with anvi'o. Nat Microbiol 6, 3-6. 10.1038/s41564-020-00834-3.
29. Sekulovic, O., Meessen-Pinard, M., and Fortier, L.-C. (2011). Prophage-stimulated toxin production in Clostridium difficile NAP1/027 lysogens. J Bacteriol 193, 2726-2734. 10.1128/JB.00787-10.
30. Sekulovic, O., Ospina Bedoya, M., Fivian-Hughes, A. S., Fairweather, N. F., and Fortier, L.-C. (2015). The Clostridium difficile cell wall protein CwpV confers phase-variable phage resistance. Mol Microbiol 98, 329-342. 10.1111/mmi.13121.
31. Nale, J. Y., Spencer, J., Hargreaves, K. R., Buckley, A. M., Trzepiński, P., Douce, G. R., and Clokie, M. R. J. (2016). Bacteriophage Combinations Significantly Reduce Clostridium difficile Growth In Vitro and Proliferation In Vivo. Antimicrobial Agents and Chemotherapy 60, 968-981. 10.1128/AAC.01774-15.
32. Shan, J., Ramachandran, A., Thanki, A. M., Vukusic, F. B. I., Barylski, J., and Clokic, M. R. J. (2018). Bacteriophages are more virulent to bacteria with human cells than they are in bacterial culture; insights from HT-29 cells. Sci Rep 8, 5091. 10.1038/s41598-018-23418-y.
33. Fortier, L.-C. (2018). Bacteriophages Contribute to Shaping Clostridioides (Clostridium) difficile Species. Front Microbiol 9, 2033. 10.3389/fmich.2018.02033.
34. Heuler, J., Fortier, L.-C., and Sun, X. (2021). Clostridioides difficile phage biology and application. FEMS Microbiol Rev 45, fuab012. 10.1093/femsre/fuab012.
35. Carter, G. P., Lyras, D., Allen, D. L., Mackin, K. E., Howarth, P. M., O'Connor, J. R., and Rood, J. I. (2007). Binary toxin production in Clostridium difficile is regulated by CdtR, a LytTR family response regulator. J Bacteriol 189, 7290-7301. 10.1128/JB.00731-07.
36. Govind, R., and Dupuy, B. (2012). Secretion of Clostridium difficile toxins A and B requires the holin-like protein TcdE. PLOS Pathog 8, e1002727. 10.1371/journal.ppat. 1002727.
37. Tan, K. S., Wec, B. Y., and Song. K. P. (2001). Evidence for holin function of tcdE gene in the pathogenicity of Clostridium difficile. J Med Microbiol 50, 613-619. 10.1099/0022-1317-50-7-613.
38. Govind, R., Fitzwater, L., and Nichols, R. (2015). Observations on the Role of TedE Isoforms in Clostridium difficile Toxin Secretion. J Bacteriol 197, 2600-2609. 10.1128/JB.00224-15.
39. Dupuy, B., Raffestin, S., Matamouros, S., Mani, N., Popoff, M. R., and Sonenshein, A. L. (2006). Regulation of toxin and bacteriocin gene expression in Clostridium by interchangeable RNA polymerase sigma factors. Mol Microbiol 60, 1044-1057. 10.1111/j.1365-2958.2006.05159.x.
40. Mani, N., Lyras, D., Barroso, L., Howarth, P., Wilkins, T., Rood, J. I., Sonenshein, A. L., and Dupuy, B. (2002). Environmental response and autoregulation of Clostridium difficile TxeR, a sigma factor for toxin gene expression. J Bacteriol 184, 5971-5978. 10.1128/JB.184.21.5971-5978.2002.
41. Eyre, D. W., Cule, M. L., Wilson, D. J., Griffiths, D., Vaughan, A., O'Connor, L., Ip, C. L. C., Golubchik, T., Batty, E. M., Finney, J. M., et al. (2013). Diverse sources of C. difficile infection identified on whole-genome sequencing. N Engl J Med 369, 1195-1205. 10.1056/NEJMoa1216064.
42. Bilverstone, T. W., Minton, N. P., and Kuchne, S. A. (2019). Phosphorylation and functionality of CdtR in Clostridium difficile. Anaerobe 58, 103-109. 10.1016/j.anaerobc.2019.102074.
43. Chen, R., Feng. Y., Wang, X., Yang, J., Zhang, X., Lü, X., and Zong, Z. (2017). Whole genome sequences of three Clade 3 Clostridium difficile strains carrying binary toxin genes in China. Sci Rep 7, 43555. 10.1038/srep43555.
44. Barbut, F., Decré, D., Lalande, V., Burghoffer, B., Noussair, L., Gigandon, A., Espinasse, F., Raskine, L., Robert, J., Mangcol, A., et al. (2005). Clinical features of Clostridium difficile-associated diarrhoca due to binary toxin (actin-specific ADP-ribosyltransferase)-producing strains. J Med Microbiol 54, 181-185. 10.1099/jmm.0.45804-0.
45. Young, M. K., Leslie, J. L., Madden, G. R., Lyerly, D. M., Carman, R. J., Lyerly, M. W., Stewart, D. B., Abhyankar, M. M., and Petri, W. A. (2022). Binary Toxin Expression by Clostridioides difficile Is Associated With Worse Disease. Open Forum Infect Dis 9, ofac001. 10.1093/ofid/ofac001.
46. Carter, G. P., Chakravorty, A., Pham Nguyen, T. A., Mileto, S., Schreiber, F., Li, L., Howarth, P., Clare, S., Cunningham, B., Sambol, S. P., et al. (2015). Defining the Roles of TedA and TcdB in Localized Gastrointestinal Disease, Systemic Organ Damage, and the Host Response during Clostridium difficile Infections. mBio 6, c00551. 10.1128/mBio.00551-15.
47. Cowardin, C. A., Buonomo, E. L., Salch, M. M., Wilson, M. G., Burgess, S. L., Kuchne, S. A., Schwan, C., Eichhoff. A. M., Koch-Nolte, F., Lyras, D., et al. (2016). The binary toxin CDT enhances Clostridium difficile virulence by suppressing protective colonic cosinophilia. Nat Microbiol l, 16108. 10.1038/nmicrobiol.2016.108.
48. Metcalf, D. S., and Weese, J. S. (2011). Binary toxin locus analysis in Clostridium difficile. J Med Microbiol 60, 1137-1145. 10.1099/jmm.0.028498-0.
49. Kuchne, S. A., Cartman, S. T., Heap, J. T., Kelly, M. L., Cockayne, A., and Minton, N. P. (2010). The role of toxin A and toxin B in Clostridium difficile infection. Nature 467, 711-713. 10.1038/nature09397.
50. Alonso, C. D., Pollock, N. R., Garcy, K. W., Gonzales-Luna, A. J., Williams, D. N., Daugherty, K., Cuddemi, C., Villafuerte-Gálvez, J., White, N. C., Chen, X., et al. (2022). Higher In Vivo Fecal Concentrations of Clostridioides difficile Toxins A and B in Patients With North American Pulsed-Field Gel Electrophoresis Type 1/Ribotype 027 Strain Infection. Clin Infect Dis 75, 2019-2022. 10.1093/cid/ciac406.
51. Alonso, C. D., Kelly, C. P., Garey, K. W., Gonzales-Luna, A. J., Williams, D., Daugherty, K., Cuddemi, C., Villafuerte-Gálvez, J., White, N. C., Chen, X., et al. (2022). Ultrasensitive and Quantitative Toxin Measurement Correlates With Baseline Severity, Severe Outcomes, and Recurrence Among Hospitalized Patients With Clostridioides difficile Infection. Clin Infect Dis 74, 2142-2149. 10.1093/cid/ciab826.
52. Guh, A. Y., Hatfield, K. M., Winston, L. G., Martin, B., Johnston, H., Brousseau, G., Farley, M. M., Wilson, L., Perlmutter, R., Phipps, E. C., et al. (2019). Toxin Enzyme Immunoassays Detect Clostridioides difficile Infection With Greater Severity and Higher Recurrence Rates. Clinical Infectious Diseases 69, 1667-1674. 10.1093/cid/ciz009.
53. Gálvez, J. A. V., Pollock, N. R., Alonso, C. D., Chen, X., Xu, H., Wang, L., White, N., Banz, A., Miller, M., Daugherty, K., et al. (2022). Stool interleukin-1ß differentiates Clostridioides difficile infection from asymptomatic carriage and non-C. difficile infection diarrhea. Clin Infect Dis, ciac624. 10.1093/cid/ciac624.
54. Govind, R., Vediyappan, G., Rolfe, R. D., Dupuy, B., and Fralick, J. A. (2009). Bacteriophage-Mediated Toxin Gene Regulation in Clostridium difficile. J Virol 83, 12037-12045. 10.1128/JVI.01256-09.
55. Goh, S., Chang, B. J., and Riley, T. V. (2005). Effect of phage infection on toxin production by Clostridium difficile. J Med Microbiol 54, 129-135. 10.1099/jmm.0.45821-0.
56. Hargreaves, K. R., Kropinski, A. M., and Clokic, M. R. J. (2014). What does the talking?: quorum sensing signalling genes discovered in a bacteriophage genome. PLOS One 9, c85131. 10.1371/journal.pone.0085131.
57. Nale, J. Y., Chutia, M., Carr, P., Hickenbotham, P. T., and Clokic, M. R. J. (2016). “Get in Early”; Biofilm and Wax Moth (Galleria mellonella) Models Reveal New Insights into the Therapeutic Potential of Clostridium difficile Bacteriophages. Front Microbiol 7, 1383. 10.3389/fmich.2016.01383.
58. Griffiths, D., Fawley, W., Kachrimanidou, M., Bowden, R., Crook, D. W., Fung, R., Golubchik, T., Harding, R. M., Jeffery, K. J. M., Jolley, K. A., et al. (2010). Multilocus Sequence Typing of Clostridium difficile. Journal of Clinical Microbiology 48, 770-778. 10.1128/JCM.01796-09.
59. Sorg. J. A., and Sonenshein, A. L. (2008). Bile salts and glycine as cogerminants for Clostridium difficile spores. J Bacteriol 190, 2505-2512. 10.1128/JB.01765-07.
60. Abt, M. C., Lewis, B. B., Caballero, S., Xiong, H., Carter, R. A., Sušac, B., Ling. L., Leiner, I., and Pamer, E. G. (2015). Innate Immune Defenses Mediated by Two ILC Subsets Are Critical for Protection against Acute Clostridium difficile Infection. Cell Host Microbe 18, 27-37. 10.1016/j.chom.2015.06.011.
61. Keith, J. W., Dong, Q., Sorbara, M. T., Becattini, S., Sia, J. K., Gjonbalaj, M., Scok, R., Leiner, I. M., Littmann, E. R., and Pamer, E. G. (2020). Impact of Antibiotic-Resistant Bacteria on Immune Activation and Clostridioides difficile Infection in the Mouse Intestine. Infect Immun 88, c00362-19. 10.1128/IAI.00362-19.
62. Kaus, G. M., Snyder, L. F., Müh, U., Flores, M. J., Popham, D. L., and Ellermeier, C. D. (2020). Lysozyme Resistance in Clostridioides difficile Is Dependent on Two Peptidoglycan Deacetylases. J Bacteriol 202, c00421-20. 10.1128/JB.00421-20.
63. Edwards, A. N., Krall, E. G., and McBride, S. M. (2020). Strain-Dependent RstA Regulation of Clostridioides difficile Toxin Production and Sporulation. J Bacteriol 202, c00586-19. 10.1128/JB.00586-19.
64. Angione, S. L., Sarma, A. A., Novikov, A., Seward, L., Fieber, J. H., Mermel, L. A., and Tripathi, A. (2014). A novel subtyping assay for detection of Clostridium difficile virulence genes. J Mol Diagn 16, 244-252. 10.1016/j.jmoldx.2013.11.006.
65. Babakhani, F., Bouillaut, L., Scars, P., Sims, C., Gomez, A., and Sonenshein, A. L. (2013). Fidaxomicin inhibits toxin production in Clostridium difficile. Journal of Antimicrobial Chemotherapy 68, 515-522. 10.1093/jac/dks450.
66. Wroblewski, D., Hannett, G. E., Bopp, D. J., Dumyati, G. K., Halse, T. A., Dumas, N. B., and Musser, K. A. (2009). Rapid molecular characterization of Clostridium difficile and assessment of populations of C. difficile in stool specimens. J Clin Microbiol 47, 2142-2148. 10.1128/JCM.02498-08.
67. Metcalf. D., Sharif, S., and Weese, J. S. (2010). Evaluation of candidate reference genes in Clostridium difficile for gene expression normalization. Anaerobe 16, 439-443. 10.1016/j.anaerobe.2010.06.007.
68. McAllister, K. N., Bouillaut, L., Kahn, J. N., Self, W. T., and Sorg. J. A. (2017). Using CRISPR-Cas9-mediated genome editing to generate C. difficile mutants defective in selenoproteins synthesis. Sci Rep 7, 14672. 10.1038/s41598-017-15236-5.
69. Prjibelski, A., Antipov, D., Meleshko, D., Lapidus, A., and Korobeynikov, A. (2020). Using SPAdes De Novo Assembler. Curr Protoc Bioinformatics 70, e102. 10.1002/cpbi.102.
70. Seemann, T. (2014). Prokka: rapid prokaryotic genome annotation. Bioinformatics 30, 2068-2069. 10.1093/bioinformatics/btu153.
71. Wick, R. R., Judd, L. M., Gorrie, C. L., and Holt, K. E. (2017). Unicycler: Resolving bacterial genome assemblies from short and long sequencing reads. PLOS Comput Biol 13, e1005595. 10.1371/journal.pcbi. 1005595.
72. Camacho, C., Coulouris, G., Avagyan, V., Ma, N., Papadopoulos, J., Bealer, K., and Madden, T. L. (2009). BLAST+: architecture and applications. BMC Bioinformatics 10, 421. 10.1186/1471-2105-10-421.
73. Seemann, T. (2023). mlst.
74. Li, W., and Godzik, A. (2006). Cd-hit: a fast program for clustering and comparing large sets of protein or nucleotide sequences. Bioinformatics 22, 1658-1659. 10.1093/bioinformatics/bt1158.
75. L. F., B. N., Z. Z., S. W., and W. L. (2012). CD-HIT: accelerated for clustering the next-generation sequencing data. Bioinformatics (Oxford, England) 28. 10.1093/bioinformatics/bts565.
76. Seemann. T. (2023). Snippy.
77. Croucher, N. J., Page, A. J., Connor, T. R., Delaney, A. J., Keane, J. A., Bentley, S. D., Parkhill, J., and Harris, S. R. (2015). Rapid phylogenetic analysis of large samples of recombinant bacterial whole genome sequences using Gubbins. Nucleic Acids Res 43, e15. 10.1093/nar/gku1196.
78. Price, M. N., Dehal, P. S., and Arkin, A. P. (2009). FastTree: computing large minimum evolution trees with profiles instead of a distance matrix. Mol Biol Evol 26, 1641-1650. 10.1093/molbev/msp077.
79. Price, M. N., Dehal, P. S., and Arkin, A. P. (2010). FastTree 2—approximately maximum-likelihood trees for large alignments. PLOS One 5, e9490. 10.1371/journal.pone.0009490.
80. Jain, C., Rodriguez-R, L. M., Phillippy, A. M., Konstantinidis, K. T., and Aluru, S. (2018). High throughput ANI analysis of 90K prokaryotic genomes reveals clear species boundaries. Nat Commun 9, 5114. 10.1038/s41467-018-07641-9.
81. Buchfink, B., Xie, C., and Huson, D. H. (2015). Fast and sensitive protein alignment using DIAMOND. Nat Methods 12, 59-60. 10.1038/nmeth.3176.
82. van Dongen, S., and Abreu-Goodger, C. (2012). Using MCL to extract clusters from networks. Methods Mol Biol 804, 281-295. 10.1007/978-1-61779-361-5_15.
83. Benedict, M. N., Henriksen, J. R., Metcalf, W. W., Whitaker, R. J., and Price, N. D. (2014). ITEP: An integrated toolkit for exploration of microbial pan-genomes. BMC Genomics 15, 8. 10.1186/1471-2164-15-8.
84. Galperin, M. Y., Makarova, K. S., Wolf, Y. I., and Koonin, E. V. (2015). Expanded microbial genome coverage and improved protein family annotation in the COG database. Nucleic Acids Res 43, D261-269. 10.1093/nar/gku1223.
85. Arndt, D., Grant, J. R., Marcu, A., Sajed, T., Pon, A., Liang, Y., and Wishart, D. S. (2016). PHASTER: a better, faster version of the PHAST phage search tool. Nucleic Acids Res 44, W16-21. 10.1093/nar/gkw387.
86. Sekulovic, O., and Fortier, L.-C. (2015). Global transcriptional response of Clostridium difficile carrying the CD38 prophage. Appl Environ Microbiol 81, 1364-1374. 10.1128/AEM.03656-14.
87. Sekulović, O., and Fortier, L.-C. (2016). Characterization of Functional Prophages in Clostridium difficile. In Clostridium difficile: Methods and Protocols Methods in Molecular Biology., A. P. Roberts and P. Mullany, eds. (Springer), pp. 143-165. 10.1007/978-1-4939-6361-4_11.

What is claimed is:

1. A method for treating and/or preventing C. difficile pathogenesis, the method comprising administering to a patient a C. difficile cell having a reduced cdtR protein expression relative to a wild type C. difficile.

2. A method for treating and/or preventing C. difficile pathogenesis, the method comprising administering to a patient a C. difficile cell that expresses a mutant cdtR protein.

3. A method for treating and/or preventing C. difficile pathogenesis, the method comprising administering to a patient a C. difficile cell that does not express a functional cdtR protein.

4. The method of any of claims 1-3, wherein the C. difficile cell has a mutant cdtR gene relative to wild type C. difficile.

5. The method of claim 4, wherein the mutant comprises a nonsense mutation.

6. The method of claim 4 or 5, where the mutant comprises a single nucleotide variation.

7. The method of any one of claims 4-6, wherein the mutant comprises a deletion.

8. The method of any of claims 1-7, wherein the C. difficile cell is an engineered C. difficile cell.

9. The method of any of claims 1-7, wherein the C. difficile cell is C. difficile ST1-75 (ATCC Deposit Number PTA-127589).

10. The method of any of claims 1-7, wherein the C. difficile cell is C. difficile ST1-35 (ATCC Deposit Number PTA-127588).

11. The method of any one of claims 1-7, wherein the C. difficile cell is C. difficile cdtRmut6.1

12. The method of any one of claims 1-7, wherein the C. difficile cell is C. difficile cdtRmut8.1

13. The method of any of claims 1-10, further comprising administering to the subject at least one additional C. difficile cell.

14. The method of claim 13, wherein the additional C. difficile cell is C. difficile ST1-75 (ATCC Deposit Number PTA-127589), C. difficile ST1-35 (ATCC Deposit Number PTA-127588), C. difficile cdtRmut6.1, or C. difficile cdtRmut8.1.

15. The method of any of claims 1-14, further comprising administering to the subject one or more antibiotics after administering the C. difficile cell.

16. A method for preventing C. difficile pathogenesis, the method comprising administering to a patient C. difficile ST1-75 (ATCC Deposit Number PTA-127589).

17. A method for preventing C. difficile pathogenesis, the method comprising administering to a patient C. difficile ST1-35 (ATCC Deposit Number PTA-127588).

18. A method for treating pathogenic C. difficile infection, the method comprising administering to a patient C. difficile ST1-75 (ATCC Deposit Number PTA-127589).

19. A method for treating pathogenic C. difficile infection, the method comprising administering to a patient C. difficile ST1-35 (ATCC Deposit Number PTA-127588).

20. A method for preventing C. difficile pathogenesis, the method comprising administering to a patient C. difficile cdtRmut6.1.

21. A method for preventing C. difficile pathogenesis, the method comprising administering to a patient C. difficile cdtRmut8.1.

22. A method for treating pathogenic C. difficile infection, the method comprising administering to a patient C. difficile cdtRmut6.1.

23. A method for treating pathogenic C. difficile infection, the method comprising administering to a patient C. difficile cdtRmut8.1.

24. A Clostridioides difficile (C. difficile) cell that expresses a mutant cdtR protein.

25. A C. difficile cell having a reduced cdtR protein expression relative to a wild type C. difficile.

26. A C. difficile cell that does not express a functional cdtR protein.

27. The C. difficile cell of any of claims 24-26, wherein the C. difficile cell has a mutant cdtR gene relative to wild type C. difficile.

28. The C. difficile cell of claim 27, wherein the mutant comprises a nonsense mutation.

29. The C. difficile cell of claim 27 or 28, where the mutant comprises a single nucleotide variation.

30. The C. difficile cell of any one of claims 27-29, wherein the mutant comprises a deletion.

31. The C. difficile cell of any of claims 24-30, wherein the C. difficile cell is an engineered C. difficile cell.

32. A method for treating and/or preventing C. difficile pathogenesis, the method comprising administering to a patient the C. difficile cell of any of claims 24-31.

33. The method of claim 32, further comprising administering to the subject one or more antibiotics after administering the C. difficile cell.

34. A composition comprising two or more of C. difficile ST1-75 (ATCC Deposit Number PTA-127589), C. difficile ST1-35 (ATCC Deposit Number PTA-127588), C. difficile cdtRmut6.1, and C. difficile cdtRmut8.1.

35. A method for analyzing virulence of a bacteria strain present in an infection in a patient, the method comprising identifying a cdtR gene in the bacteria strain.

36. The method of claim 35, wherein the bacteria strain is a C. difficile bacteria strain.

37. The method of claim 35 or 36, wherein the cdtR gene is a cdtR gene present in a virulent bacteria strain.

38. The method of any one of claims 35-37, wherein the cdtR gene is a wild-type cdtR gene.

39. The method of any one of claims 35-38, wherein the C. difficile bacteria strain is R20291.

40. The method of any one of claims 35-37, wherein the cdtR gene is a mutant cdtR gene relative to a wild type cdtR gene in C. difficile.

41. The method of claim 40, wherein the mutant comprises a nonsense mutation.

42. The method of claim 40 or 41, where the mutant comprises a single nucleotide variation.

43. The method of any one of claims 40-42, wherein the mutant comprises a deletion.

44. The method of any one of claims 35-43, wherein the infection is present in the colon of the patient.

45. The method of any one of claims 35-44, wherein the identifying step comprises sequencing.

46. The method of any one of claims 35-45, wherein the identifying step comprises PCR.

47. The method of any one of claims 35-46, wherein the identifying step comprises comparing the cdtR gene identified to a standard.

48. The method of any one of claims 35-47, wherein the method further comprises administering a therapeutic intervention to the patient.

49. The method of claim 48, wherein the therapeutic intervention comprises the C. diff cell of any one of claims 24-31 or the composition of claim 34.

50. The method of claim 48 or 49, wherein the therapeutic intervention comprises an antibiotic.

51. A method of treating a bacterial infection in a patient, the method comprising administering a therapeutic intervention to the patient, wherein a cdtR gene has been identified in the bacterial infection.

52. The method of claim 51, wherein the therapeutic intervention comprises the C. diff cell of any one of claims 24-31 or the composition of claim 34.

53. The method of claim 51 or 52, wherein the therapeutic intervention comprises an antibiotic.

54. The method of any one of claims 51-53, wherein the cdtR gene identified in the bacterial infection is a wild-type cdtR gene.

55. The method of any one of claims 51-54, wherein the cdtR gene identified in the bacterial infection is a cdtR gene found in a virulent C. difficile strain.

56. The method of any one of claims 51-55, wherein the bacterial infection comprises R20291.

57. A method of reducing virulence of a bacterium, the method introducing a cdtR mutant gene to the bacterium.

58. The method of claim 57, wherein the mutant gene comprises a nonsense mutation.

59. The method of claim 57 or 58, where the mutant gene comprises a single nucleotide variation.

60. The method of any one of claims 57-59, wherein the mutant gene comprises a deletion.

61. The method of any one of claims 57-60, wherein the bacterium is a C. difficile bacterium.

62. The method of any one of claims 57-61, wherein the bacterium is R20291.

63. The method of any one of claims 57-62, wherein the cdtR mutant gene is introduced by infecting the bacterium with a bacteriophage that encodes the cdtR mutant gene.

64. The method of any one of claims 57-63, wherein the cdtR mutant gene is introduced by engineering the bacterium.

65. The method of claim 64, wherein the engineering comprises knocking out the wild-type cdtR gene.

66. The method of claim 64, wherein the engineering comprises mutating the cdtR gene.

67. The method of any one of claims 64-66, wherein the engineering comprises editing the wild-type cdtR gene by CRISPR-mediated genome editing.

68. A bacteriophage comprising an engineered genome encoding a cdtR mutant gene.

69. The bacteriophage of claim 68, wherein the mutant gene comprises a nonsense mutation.

70. The bacteriophage of claim 68 or 69, where the mutant gene comprises a single nucleotide variation.

71. The bacteriophage of any one of claims 68-70, wherein the mutant gene comprises a deletion.

72. The bacteriophage of any one of claims 68-71, wherein the bacteriophage is derived from phiCD75-2 and/or phiCD75-3.
